SEQUENCE LISTING (406 sequences)

SEQ ID NO: 1 - DNA, 24 bp, Homo sapiens
  misc_feature (1)..(1): May be labeled with a 5′ FAM fluorescent moiety
ttctgttggtatagagcagtgttt

SEQ ID NO: 2 - DNA, 19 bp, Homo sapiens
gtgacaggaaggacgaatg

SEQ ID NO: 3 - DNA, 23 bp, Homo sapiens
  misc_feature (1)..(1): May be labeled with a 5′ FAM fluorescent moiety
attttccccgatgatagtagtct

SEQ ID NO: 4 - DNA, 26 bp, Homo sapiens
gcgaatgtatgattggcaatattttt

SEQ ID NO: 5 - DNA, 25 bp, Homo sapiens
  misc_feature (1)..(1): May be labeled with a 5′ FAM fluorescent moiety
ttaatgaattgaacaaatgagtgag

SEQ ID NO: 6 - DNA, 25 bp, Homo sapiens
gcaactctggttgtattgtcttcat

SEQ ID NO: 7 - DNA, 20 bp, Homo sapiens
  misc_feature (1)..(1): May be labeled with a 5′ TAMRA fluorescent moiety
tcacctgtcccagctatctg

SEQ ID NO: 8 - DNA, 25 bp, Homo sapiens
aaaattactggtacacttgatagct

SEQ ID NO: 9 - DNA, 22 bp, Homo sapiens
  misc_feature (1)..(1): May be labeled with a 5′ NED fluorescent moiety
cttagggaaccctcactgaatg

SEQ ID NO: 10 - DNA, 22 bp, Homo sapiens
gtccttgtcagcgtttatttgc

SEQ ID NO: 11 - DNA, 20 bp, Homo sapiens
  misc_feature (1)..(1): May be labeled with a 5′ FAM fluorescent moiety
cacaaggcagaataaaggga

SEQ ID NO: 12 - DNA, 26 bp, Homo sapiens
ttcatagtctgtcttgtcttgtctca

SEQ ID NO: 13 - DNA, 20 bp, Homo sapiens
  misc_feature (1)..(1): May be labeled with a 5′ TAMRA fluorescent moiety
caccatacccagccttactg

SEQ ID NO: 14 - DNA, 27 bp, Homo sapiens
catgttactgtgctgaatattgtaggc

SEQ ID NO: 15 - DNA, 20 bp, Homo sapiens
  misc_feature (1)..(1): May be labeled with a 5′ 6-FAM fluorescent moiety
ttgagaaggccccacctatg

SEQ ID NO: 16 - DNA, 20 bp, Homo sapiens
agaaggccccctgtgttatg

SEQ ID NO: 17 - DNA, 23 bp, Homo sapiens
  misc_feature (1)..(1): May be labeled with a 5′ HEX fluorescent moiety
gcgaatcatgacactaattttgg

SEQ ID NO: 18 - DNA, 22 bp, Homo sapiens
tggagaagaaaaagaggcctga

SEQ ID NO: 19 - DNA, 24 bp, Homo sapiens
  misc_feature (1)..(1): May be labeled with a 5′ NED fluorescent moiety
ggttctttggaaaattgtttaggc

SEQ ID NO: 20 - DNA, 22 bp, Homo sapiens
tgctctggacttacagcatcaa

SEQ ID NO: 21 - DNA, 25 bp, Homo sapiens
  misc_feature (1)..(1): May be labeled with a 5′ 6-FAM fluorescent moiety
cccttaaaaccatatttttcacctc

SEQ ID NO: 22 - DNA, 20 bp, Homo sapiens
agcctgggtgacagagcaag

SEQ ID NO: 23 - DNA, 23 bp, Homo sapiens
  misc_feature (1)..(1): May be labeled with a 5′ HEX fluorescent moiety
ttgcttgagaggtaaaaagaaaa

SEQ ID NO: 24 - DNA, 21 bp, Homo sapiens
gagcaacagagcgagattctg

SEQ ID NO: 25 - DNA, 20 bp, Homo sapiens
  misc_feature (1)..(1): May be labeled with a 5′ NED fluorescent moiety
caccatacccagccttactg

SEQ ID NO: 26 - DNA, 27 bp, Homo sapiens
catgttactgtgctgaatattgtaggc

SEQ ID NO: 27 - DNA, 22 bp, Homo sapiens
  misc_feature (1)..(1): May be labeled with a 5′ 6-FAM fluorescent moiety
ggcagaataaagggattattgc

SEQ ID NO: 28 - DNA, 26 bp, Homo sapiens
ttcatagtctgtcttgtcttgtctca

SEQ ID NO: 29 - DNA, 24 bp, Homo sapiens
  misc_feature (1)..(1): May be labeled with a 5′ NED fluorescent moiety
gcagtaaatgtctatgaaacaagg

SEQ ID NO: 30 - DNA, 27 bp, Homo sapiens
tgataaatagtgaatatagttgacagc

SEQ ID NO: 31 - DNA, 23 bp, Homo sapiens
  misc_feature (1)..(1): May be labeled with a 5′ 6-FAM, VIC, NED, JOE, ROX, PET, TAMRA or 5-FAM fluorescent moiety
caacaattccttctaattttcca

SEQ ID NO: 32 - DNA, 20 bp, Homo sapiens
tgtctggtttccccatctct

SEQ ID NO: 33 - DNA, 22 bp, Homo sapiens
  misc_feature (1)..(1): May be labeled with a 5′ 6-FAM, VIC, NED, JOE, ROX, PET, TAMRA or 5-FAM fluorescent moiety
tgaggagacatccttgacaaaa

SEQ ID NO: 34 - DNA, 24 bp, Homo sapiens
catacacacagcaagtatgagtga

SEQ ID NO: 35 - DNA, 21 bp, Homo sapiens
  misc_feature (1)..(1): May be labeled with a 5′ 6-FAM, VIC, NED, JOE, ROX, PET, TAMRA or 5-FAM fluorescent moiety
gcctatggtcccatcataaca

SEQ ID NO: 36 - DNA, 22 bp, Homo sapiens
tccacagttcttagatggcttt

SEQ ID NO: 37 - DNA, 22 bp, Homo sapiens
  misc_feature (1)..(1): May be labeled with a 5′ 6-FAM, VIC, NED, JOE, ROX, PET, TAMRA or 5-FAM fluorescent moiety
tgaaaacgtgttgacagatgaa

SEQ ID NO: 38 - DNA, 20 bp, Homo sapiens
aatggcaggattttcttttt

SEQ ID NO: 39 - DNA, 26 bp, Homo sapiens
  misc_feature (1)..(1): May be labeled with a 5′ 6-FAM, VIC, NED, JOE, ROX, PET, TAMRA or 5-FAM fluorescent moiety
tgtcaaaatatgtggattagacaaaa

SEQ ID NO: 40 - DNA, 24 bp, Homo sapiens
tcactggtatactgtgatgtgtgc

SEQ ID NO: 41 - DNA, 25 bp, Homo sapiens
  misc_feature (1)..(1): May be labeled with a 5′ 6-FAM, VIC, NED, JOE, ROX, PET, TAMRA or 5-FAM fluorescent moiety
aaaaattattgataacgttcagtat

SEQ ID NO: 42 - DNA, 25 bp, Homo sapiens
tttctaacaatatgtacccactgga

SEQ ID NO: 43 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
cattgggccttctgtcaaat

SEQ ID NO: 44 - DNA, 22 bp, Homo sapiens
tctgcagaatttcatttgctgt

SEQ ID NO: 45 - DNA, 27 bp, Homo sapiens
  misc_feature (1)..(1): May be labeled with a 5′ 6-FAM, VIC, NED, JOE, ROX, PET, TAMRA or 5-FAM fluorescent moiety
caaacttgaaatatgaaaaagtcatca

SEQ ID NO: 46 - DNA, 26 bp, Homo sapiens
tttctgtatttcctgaaacaacattt

SEQ ID NO: 47 - DNA, 26 bp, Homo sapiens
  misc_feature (1)..(1): May be labeled with a 5′ 6-FAM, VIC, NED, JOE, ROX, PET, TAMRA or 5-FAM fluorescent moiety
tctgtctttgtctcactatctgtctg

SEQ ID NO: 48 - DNA, 20 bp, Homo sapiens
tagggtgaggacccctttct

SEQ ID NO: 49 - DNA, 24 bp, Homo sapiens
  misc_feature (1)..(1): May be labeled with a 5′ 6-FAM, VIC, NED, JOE, ROX, PET, TAMRA or 5-FAM fluorescent moiety
cctggattatttgttcaaagtcag

SEQ ID NO: 50 - DNA, 20 bp, Homo sapiens
tctcatgttccttggcctgt

SEQ ID NO: 51 - DNA, 23 bp, Homo sapiens
  misc_feature (1)..(1): May be labeled with a 5′ 6-FAM, VIC, NED, JOE, ROX, PET, TAMRA or 5-FAM fluorescent moiety
attttgccacatagagaaaaata

SEQ ID NO: 52 - DNA, 19 bp, Homo sapiens
gcctggacatcctctttct

SEQ ID NO: 53 - DNA, 24 bp, Homo sapiens
  misc_feature (1)..(1): May be labeled with a 5′ 6-FAM, VIC, NED, JOE, ROX, PET, TAMRA or 5-FAM fluorescent moiety
agatgtagcacagttagatgcaga

SEQ ID NO: 54 - DNA, 20 bp, Homo sapiens
agcagaaaaagccattctgg

SEQ ID NO: 55 - DNA, 18 bp, Homo sapiens
  misc_feature (1)..(1): May be labeled with a 5′ 6-FAM, VIC, NED, JOE, ROX, PET, TAMRA or 5-FAM fluorescent moiety
gtcatgaccctggctgtg

SEQ ID NO: 56 - DNA, 18 bp, Homo sapiens
agggcaggctgtgctcat

SEQ ID NO: 57 - DNA, 23 bp, Homo sapiens
  misc_feature (1)..(1): May be labeled with a 5′ 6-FAM, VIC, NED, JOE, ROX, PET, TAMRA or 5-FAM fluorescent moiety
gggaccattttagatattctgct

SEQ ID NO: 58 - DNA, 23 bp, Homo sapiens
gcacttaacaagcactgaatcaa

SEQ ID NO: 59 - DNA, 21 bp, Homo sapiens
  misc_feature (1)..(1): May be labeled with a 5′ 6-FAM, VIC, NED, JOE, ROX, PET, TAMRA or 5-FAM fluorescent moiety
tcctgaaggaagaatgtggtc

SEQ ID NO: 60 - DNA, 19 bp, Homo sapiens
atgcatggtcgtgtgtgtg

SEQ ID NO: 61 - DNA, 25 bp, Homo sapiens
  misc_feature (1)..(1): May be labeled with a 5′ 6-FAM, VIC, NED, JOE, ROX, PET, TAMRA or 5-FAM fluorescent moiety
tttttcaaaatcaaaaagatagtga

SEQ ID NO: 62 - DNA, 20 bp, Homo sapiens
ggagggcatctgggtaattt

SEQ ID NO: 63 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
gccatcagccctcatacaga

SEQ ID NO: 64 - DNA, 18 bp, Homo sapiens
gaatttgggggacgcagt

SEQ ID NO: 65 - DNA, 21 bp, Homo sapiens
  misc_feature (1)..(1): May be labeled with a 5′ 6-FAM, VIC, NED, JOE, ROX, PET, TAMRA or 5-FAM fluorescent moiety
cgagaagtttcccatgcattt

SEQ ID NO: 66 - DNA, 22 bp, Homo sapiens
aaattcagtgatgggaagaagg

SEQ ID NO: 67 - DNA, 22 bp, Homo sapiens
  misc_feature (1)..(1): May be labeled with a 5′ 6-FAM, VIC, NED, JOE, ROX, PET, TAMRA or 5-FAM fluorescent moiety
cctaaaacagcatcaacagaaa

SEQ ID NO: 68 - DNA, 21 bp, Homo sapiens
ttggaccttttgatttttcct

SEQ ID NO: 69 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
cagcaactcccacttctgac

SEQ ID NO: 70 - DNA, 27 bp, Homo sapiens
ttgttgttgagtgaaagaatagagaaa

SEQ ID NO: 71 - DNA, 25 bp, Homo sapiens
  misc_feature (1)..(1): May be labeled with a 5′ 6-FAM, VIC, NED, JOE, ROX, PET, TAMRA or 5-FAM fluorescent moiety
aaatctgaattatgtccaatcaaaa

SEQ ID NO: 72 - DNA, 25 bp, Homo sapiens
tgagtttttggaagagagagagaga

SEQ ID NO: 73 - DNA, 21 bp, Homo sapiens
  misc_feature (1)..(1): May be labeled with a 5′ 6-FAM, VIC, NED, JOE, ROX, PET, TAMRA or 5-FAM fluorescent moiety
aaaaggggatccaatatgaaa

SEQ ID NO: 74 - DNA, 26 bp, Homo sapiens
tggaacaattttatccttagtttgtc

SEQ ID NO: 75 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
tatgcacaccacacggacac

SEQ ID NO: 76 - DNA, 20 bp, Homo sapiens
gttccggggaagttttatgc

SEQ ID NO: 77 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
tggcaaagagaacaacagca

SEQ ID NO: 78 - DNA, 22 bp, Homo sapiens
ttctgtgaatatgggttctgga

SEQ ID NO: 79 - DNA, 26 bp, Homo sapiens
  misc_feature (1)..(1): May be labeled with a 5′ 6-FAM, VIC, NED, JOE, ROX, PET, TAMRA or 5-FAM fluorescent moiety
ggggattatttatgtagaaaatgaga

SEQ ID NO: 80 - DNA, 23 bp, Homo sapiens
ggtgacagaccctgtctctaaaa

SEQ ID NO: 81 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
tgggcaacagagtgagacag

SEQ ID NO: 82 - DNA, 20 bp, Homo sapiens
cacatccttgccaacacttg

SEQ ID NO: 83 - DNA, 24 bp, Homo sapiens
  misc_feature (1)..(1): May be labeled with a 5′ 6-FAM, VIC, NED, JOE, ROX, PET, TAMRA or 5-FAM fluorescent moiety
gatactgaatcatccctttcattc

SEQ ID NO: 84 - DNA, 25 bp, Homo sapiens
ttccgttattaattttattctgagg

SEQ ID NO: 85 - DNA, 24 bp, Homo sapiens
  misc_feature (1)..(1): May be labeled with a 5′ 6-FAM, VIC, NED, JOE, ROX, PET, TAMRA or 5-FAM fluorescent moiety
gagtgagagacagacagagagacg

SEQ ID NO: 86 - DNA, 24 bp, Homo sapiens
cagtaggggcagactattttactc

SEQ ID NO: 87 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
atgggtgacagagcgagact

SEQ ID NO: 88 - DNA, 20 bp, Homo sapiens
ttcagagcctgggttaaaca

SEQ ID NO: 89 - DNA, 24 bp, Homo sapiens
  misc_feature (1)..(1): May be labeled with a 5′ 6-FAM, VIC, NED, JOE, ROX, PET, TAMRA or 5-FAM fluorescent moiety
aatgaccatccttaaaacaacttt

SEQ ID NO: 90 - DNA, 20 bp, Homo sapiens
gtggctgagcgagactctgt

SEQ ID NO: 91 - DNA, 23 bp, Homo sapiens
  misc_feature (1)..(1): May be labeled with a 5′ 6-FAM, VIC, NED, JOE, ROX, PET, TAMRA or 5-FAM fluorescent moiety
tcactcaatttatgagagcgaaa

SEQ ID NO: 92 - DNA, 21 bp, Homo sapiens
tcagcctttgatatgtgcatt

SEQ ID NO: 93 - DNA, 22 bp, Homo sapiens
  misc_feature (1)..(1): May be labeled with a 5′ 6-FAM, VIC, NED, JOE, ROX, PET, TAMRA or 5-FAM fluorescent moiety
gcatgagtggaaaaatgtgaaa

SEQ ID NO: 94 - DNA, 21 bp, Homo sapiens
ctgagtcaagagcaggcaact

SEQ ID NO: 95 - DNA, 22 bp, Homo sapiens
  misc_feature (1)..(1): May be labeled with a 5′ 6-FAM, VIC, NED, JOE, ROX, PET, TAMRA or 5-FAM fluorescent moiety
ccaatgcttttgatttttaagc

SEQ ID NO: 96 - DNA, 24 bp, Homo sapiens
cggcaaagtagtatttaatgtgct

SEQ ID NO: 97 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
tttcattcaccggttttcct

SEQ ID NO: 98 - DNA, 25 bp, Homo sapiens
ttttagggtatatctgccataatgc

SEQ ID NO: 99 - DNA, 22 bp, Homo sapiens
  misc_feature (1)..(1): May be labeled with a 5′ 6-FAM, VIC, NED, JOE, ROX, PET, TAMRA or 5-FAM fluorescent moiety
gggaaggaaaataaagcattga

SEQ ID NO: 100 - DNA, 23 bp, Homo sapiens
gcacaaaaacaaaatctgtcact

SEQ ID NO: 101 - DNA, 24 bp, Homo sapiens
  misc_feature (1)..(1): May be labeled with a 5′ 6-FAM, VIC, NED, JOE, ROX, PET, TAMRA or 5-FAM fluorescent moiety
ttgctgggttgttaaacttattca

SEQ ID NO: 102 - DNA, 20 bp, Homo sapiens
aaaggtgaacgctggtatcg

SEQ ID NO: 103 - DNA, 19 bp, Homo sapiens
  misc_feature (1)..(1): May be labeled with a 5′ 6-FAM, VIC, NED, JOE, ROX, PET, TAMRA or 5-FAM fluorescent moiety
gaccccaacactggacaca

SEQ ID NO: 104 - DNA, 20 bp, Homo sapiens
cttggtaagtgggcagtgag

SEQ ID NO: 105 - DNA, 22 bp, Homo sapiens
  misc_feature (1)..(1): May be labeled with a 5′ 6-FAM, VIC, NED, JOE, ROX, PET, TAMRA or 5-FAM fluorescent moiety
catgctcatagatatgcacaca

SEQ ID NO: 106 - DNA, 20 bp, Homo sapiens
gattgtgccaggtctcaggt

SEQ ID NO: 107 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
ccaaggtgaattcccaattt

SEQ ID NO: 108 - DNA, 20 bp, Homo sapiens
gcggcacattttcacagact

SEQ ID NO: 109 - DNA, 27 bp, Homo sapiens
  misc_feature (1)..(1): May be labeled with a 5′ 6-FAM, VIC, NED, JOE, ROX, PET, TAMRA or 5-FAM fluorescent moiety
tgaacattaacactggtaactttacat

SEQ ID NO: 110 - DNA, 20 bp, Homo sapiens
tgtcctctccattttgcttg

SEQ ID NO: 111 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
cttctctccaattcccatgc

SEQ ID NO: 112 - DNA, 21 bp, Homo sapiens
gaaggaaaaattgggatttcg

SEQ ID NO: 113 - DNA, 19 bp, Homo sapiens
  misc_feature (1)..(1): May be labeled with a 5′ 6-FAM, VIC, NED, JOE, ROX, PET, TAMRA or 5-FAM fluorescent moiety
cagcctgggtgacagagac

SEQ ID NO: 114 - DNA, 26 bp, Homo sapiens
tgttgatgaagcatttactcatacat

SEQ ID NO: 115 - DNA, 21 bp, Homo sapiens
  misc_feature (1)..(1): May be labeled with a 5′ 6-FAM, VIC, NED, JOE, ROX, PET, TAMRA or 5-FAM fluorescent moiety
tccccctaaatggacaacttt

SEQ ID NO: 116 - DNA, 22 bp, Homo sapiens
gctttgttttcctttagcttcc

SEQ ID NO: 117 - DNA, 21 bp, Homo sapiens
  misc_feature (1)..(1): May be labeled with a 5′ 6-FAM, VIC, NED, JOE, ROX, PET, TAMRA or 5-FAM fluorescent moiety
tctggaatggttaaggcagaa

SEQ ID NO: 118 - DNA, 20 bp, Homo sapiens
caccattctccctagcatga

SEQ ID NO: 119 - DNA, 19 bp, Homo sapiens
  misc_feature (1)..(1): May be labeled with a 5′ 6-FAM, VIC, NED, JOE, ROX, PET, TAMRA or 5-FAM fluorescent moiety
caaggtggaaggcagaagg

SEQ ID NO: 120 - DNA, 20 bp, Homo sapiens
cgcgctctctctctctctct

SEQ ID NO: 121 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
tgcaagatttcccccttcta

SEQ ID NO: 122 - DNA, 20 bp, Homo sapiens
ctttgactcctcagccgtgt

SEQ ID NO: 123 - DNA, 19 bp, Homo sapiens
  misc_feature (1)..(1): May be labeled with a 5′ 6-FAM, VIC, NED, JOE, ROX, PET, TAMRA or 5-FAM fluorescent moiety
cctggcatctctgttttta

SEQ ID NO: 124 - DNA, 20 bp, Homo sapiens
aaagatgatgtcaggaatgc

SEQ ID NO: 125 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
catccaaggggaacacaagt

SEQ ID NO: 126 - DNA, 20 bp, Homo sapiens
ggaagtaaaaggcccagagg

SEQ ID NO: 127 - DNA, 25 bp, Homo sapiens
  misc_feature (1)..(1): May be labeled with a 5′ 6-FAM, VIC, NED, JOE, ROX, PET, TAMRA or 5-FAM fluorescent moiety
aaaaaggatgtttgttatggtaaga

SEQ ID NO: 128 - DNA, 20 bp, Homo sapiens
gaactgtgtgccaggaactg

SEQ ID NO: 129 - DNA, 24 bp, Homo sapiens
  misc_feature (1)..(1): May be labeled with a 5′ 6-FAM, VIC, NED, JOE, ROX, PET, TAMRA or 5-FAM fluorescent moiety
tgtgtgtttgcatgtatgtgtaga

SEQ ID NO: 130 - DNA, 20 bp, Homo sapiens
acagaaacatggctgcctct

SEQ ID NO: 131 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
gaccacagatttcccaatcg

SEQ ID NO: 132 - DNA, 21 bp, Homo sapiens
aaaccaactgactcccaaaca

SEQ ID NO: 133 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
cagagcgagactccatctca

SEQ ID NO: 134 - DNA, 20 bp, Homo sapiens
ggaacccttggatgttgcta

SEQ ID NO: 135 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
gctcaatgctattggagtgc

SEQ ID NO: 136 - DNA, 21 bp, Homo sapiens
ttcacaaaacagaaccaacca

SEQ ID NO: 137 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
tctcccttgccagacttctc

SEQ ID NO: 138 - DNA, 18 bp, Homo sapiens
gcccaggatggacaaaga

SEQ ID NO: 139 - DNA, 18 bp, Homo sapiens
  misc_feature (1)..(1): May be labeled with a 5′ 6-FAM, VIC, NED, JOE, ROX, PET, TAMRA or 5-FAM fluorescent moiety
cacatgtggccactgcac

SEQ ID NO: 140 - DNA, 22 bp, Homo sapiens
ccagggatttgtctagaaagga

SEQ ID NO: 141 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
gcctaggcaaccagagtgag

SEQ ID NO: 142 - DNA, 27 bp, Homo sapiens
tgtatttcttttctttttaaatggtga

SEQ ID NO: 143 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
ctcttcagcagccgagaaaa

SEQ ID NO: 144 - DNA, 20 bp, Homo sapiens
caccagaaagcaaggaagga

SEQ ID NO: 145 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
acaccagagggcagagtgag

SEQ ID NO: 146 - DNA, 24 bp, Homo sapiens
aaagcactgtatgtttctgtgaga

SEQ ID NO: 147 - DNA, 21 bp, Homo sapiens
  misc_feature (1)..(1): May be labeled with a 5′ 6-FAM, VIC, NED, JOE, ROX, PET, TAMRA or 5-FAM fluorescent moiety
ggttgtccagagaaacaacca

SEQ ID NO: 148 - DNA, 22 bp, Homo sapiens
caattgtgtgagccaatctctc

SEQ ID NO: 149 - DNA, 25 bp, Homo sapiens
  misc_feature (1)..(1): May be labeled with a 5′ 6-FAM, VIC, NED, JOE, ROX, PET, TAMRA or 5-FAM fluorescent moiety
ggcaattccttaaaataaatcactc

SEQ ID NO: 150 - DNA, 20 bp, Homo sapiens
gcccggccctatctatctat

SEQ ID NO: 151 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
gcatgctcgctctctctctt

SEQ ID NO: 152 - DNA, 21 bp, Homo sapiens
aactggcgtgtctacaccatc

SEQ ID NO: 153 - DNA, 22 bp, Homo sapiens
  misc_feature (1)..(1): May be labeled with a 5′ 6-FAM, VIC, NED, JOE, ROX, PET, TAMRA or 5-FAM fluorescent moiety
gagcaagactccatctcaaaaa

SEQ ID NO: 154 - DNA, 20 bp, Homo sapiens
aaagattgcccaacaaatgg

SEQ ID NO: 155 - DNA, 24 bp, Homo sapiens
  misc_feature (1)..(1): May be labeled with a 5′ 6-FAM, VIC, NED, JOE, ROX, PET, TAMRA or 5-FAM fluorescent moiety
tgggttccatagttctaatgtgtg

SEQ ID NO: 156 - DNA, 23 bp, Homo sapiens
tctctggctggacatactattca

SEQ ID NO: 157 - DNA, 24 bp, Homo sapiens
  misc_feature (1)..(1): May be labeled with a 5′ 6-FAM, VIC, NED, JOE, ROX, PET, TAMRA or 5-FAM fluorescent moiety
ggagaaggtagctaaaggatgaaa

SEQ ID NO: 158 - DNA, 21 bp, Homo sapiens
tgcaccctaggacctaaagaa

SEQ ID NO: 159 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
cctgatctggtcctgtagcc

SEQ ID NO: 160 - DNA, 25 bp, Homo sapiens
tgagtttgaaaataaagtgttctgc

SEQ ID NO: 161 - DNA, 21 bp, Homo sapiens
  misc_feature (1)..(1): May be labeled with a 5′ 6-FAM, VIC, NED, JOE, ROX, PET, TAMRA or 5-FAM fluorescent moiety
caaagcgagaccttttctcaa

SEQ ID NO: 162 - DNA, 24 bp, Homo sapiens
cgagtcatagatccattacccatt

SEQ ID NO: 163 - DNA, 18 bp, Homo sapiens
  misc_feature (1)..(1): May be labeled with a 5′ 6-FAM, VIC, NED, JOE, ROX, PET, TAMRA or 5-FAM fluorescent moiety
acaaccgagcgagacctg

SEQ ID NO: 164 - DNA, 24 bp, Homo sapiens
ggaactgatggtcacaagatagtc

SEQ ID NO: 165 - DNA, 21 bp, Homo sapiens
  misc_feature (1)..(1): May be labeled with a 5′ 6-FAM, VIC, NED, JOE, ROX, PET, TAMRA or 5-FAM fluorescent moiety
ttgtggattttcccaattgat

SEQ ID NO: 166 - DNA, 24 bp, Homo sapiens
tttgtatttgcctaaaaacagagc

SEQ ID NO: 167 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
taccagcttcaaacgtgcag

SEQ ID NO: 168 - DNA, 18 bp, Homo sapiens
ccttgccctgaagcacat

SEQ ID NO: 169 - DNA, 22 bp, Homo sapiens
  misc_feature (1)..(1): May be labeled with a 5′ 6-FAM, VIC, NED, JOE, ROX, PET, TAMRA or 5-FAM fluorescent moiety
tgtaggctgtttagttggtgga

SEQ ID NO: 170 - DNA, 21 bp, Homo sapiens
cggcatcacagtctgataaaa

SEQ ID NO: 171 - DNA, 26 bp, Homo sapiens
  misc_feature (1)..(1): May be labeled with a 5′ 6-FAM, VIC, NED, JOE, ROX, PET, TAMRA or 5-FAM fluorescent moiety
tgataagcctccctcactactatttt

SEQ ID NO: 172 - DNA, 24 bp, Homo sapiens
gcagcgatagctagtcatagtgaa

SEQ ID NO: 173 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
cctgcaaggacaccaagtta

SEQ ID NO: 174 - DNA, 21 bp, Homo sapiens
tgttcacctgattttcggttc

SEQ ID NO: 175 - DNA, 19 bp, Homo sapiens
  misc_feature (1)..(1): May be labeled with a 5′ 6-FAM, VIC, NED, JOE, ROX, PET, TAMRA or 5-FAM fluorescent moiety
tgcaagccacatcatttgt

SEQ ID NO: 176 - DNA, 24 bp, Homo sapiens
tcgaatcgatagatagataggtga

SEQ ID NO: 177 - DNA, 22 bp, Homo sapiens
  misc_feature (1)..(1): May be labeled with a 5′ 6-FAM, VIC, NED, JOE, ROX, PET, TAMRA or 5-FAM fluorescent moiety
tgaaacaagggaatctatcatc

SEQ ID NO: 178 - DNA, 27 bp, Homo sapiens
tgataaatagtgaatatagttgacagc

SEQ ID NO: 179 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
ttgtggggccttgtaattgt

SEQ ID NO: 180 - DNA, 21 bp, Homo sapiens
cagggtccctagagagacaga

SEQ ID NO: 181 - DNA, 23 bp, Homo sapiens
  misc_feature (1)..(1): May be labeled with a 5′ 6-FAM, VIC, NED, JOE, ROX, PET, TAMRA or 5-FAM fluorescent moiety
cagaaccaataggctatctatct

SEQ ID NO: 182 - DNA, 23 bp, Homo sapiens
acagtaaatcacttggtaggaga

SEQ ID NO: 183 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
gggcacccctttatacttgg

SEQ ID NO: 184 - DNA, 26 bp, Homo sapiens
tcacatgagccaattccttataatag

SEQ ID NO: 185 - DNA, 23 bp, Homo sapiens
  misc_feature (1)..(1): May be labeled with a 5′ 6-FAM, VIC, NED, JOE, ROX, PET, TAMRA or 5-FAM fluorescent moiety
tctatacatatgtgtgtgtgcat

SEQ ID NO: 186 - DNA, 20 bp, Homo sapiens
cacctttgttgccaagagtc

SEQ ID NO: 187 - DNA, 27 bp, Homo sapiens
  misc_feature (1)..(1): May be labeled with a 5′ 6-FAM, VIC, NED, JOE, ROX, PET, TAMRA or 5-FAM fluorescent moiety
ttctgttaaatgagtaaggagatgaca

SEQ ID NO: 188 - DNA, 20 bp, Homo sapiens
gcatgcgtgtgtgtgtgtat

SEQ ID NO: 189 - DNA, 21 bp, Homo sapiens
  misc_feature (1)..(1): May be labeled with a 5′ 6-FAM, VIC, NED, JOE, ROX, PET, TAMRA or 5-FAM fluorescent moiety
gagctgagatcacgacagtca

SEQ ID NO: 190 - DNA, 20 bp, Homo sapiens
tattttcaggccaagccttt

SEQ ID NO: 191 - DNA, 27 bp, Homo sapiens
  misc_feature (1)..(1): May be labeled with a 5′ 6-FAM, VIC, NED, JOE, ROX, PET, TAMRA or 5-FAM fluorescent moiety
gaaacagaactaataggatctatctgc

SEQ ID NO: 192 - DNA, 25 bp, Homo sapiens
ggcaaacaaatagttgatagatgag

SEQ ID NO: 193 - DNA, 26 bp, Homo sapiens
  misc_feature (1)..(1): May be labeled with a 5′ 6-FAM, VIC, NED, JOE, ROX, PET, TAMRA or 5-FAM fluorescent moiety
tgaccatcttactggtttatgtattt

SEQ ID NO: 194 - DNA, 27 bp, Homo sapiens
cgaggctattttactggtaactaactg

SEQ ID NO: 195 - DNA, 23 bp, Homo sapiens
  misc_feature (1)..(1): May be labeled with a 5′ 6-FAM, VIC, NED, JOE, ROX, PET, TAMRA or 5-FAM fluorescent moiety
gggctacatagagaaacagaacc

SEQ ID NO: 196 - DNA, 20 bp, Homo sapiens
acacacacacacacacatgc

SEQ ID NO: 197 - DNA, 23 bp, Homo sapiens
  misc_feature (1)..(1): May be labeled with a 5′ 6-FAM, VIC, NED, JOE, ROX, PET, TAMRA or 5-FAM fluorescent moiety
ggaaagagaaagaaaggaaggaa

SEQ ID NO: 198 - DNA, 25 bp, Homo sapiens
ccatttatgtcctatttcctactcc

SEQ ID NO: 199 - DNA, 21 bp, Homo sapiens
  misc_feature (1)..(1): May be labeled with a 5′ 6-FAM, VIC, NED, JOE, ROX, PET, TAMRA or 5-FAM fluorescent moiety
ggactcctccagctgaactct

SEQ ID NO: 200 - DNA, 22 bp, Homo sapiens
gcacagtacagcaaacttgtca

SEQ ID NO: 201 - DNA, 22 bp, Homo sapiens
  misc_feature (1)..(1): May be labeled with a 5′ 6-FAM, VIC, NED, JOE, ROX, PET, TAMRA or 5-FAM fluorescent moiety
tttccacacggctaatctactt

SEQ ID NO: 202 - DNA, 19 bp, Homo sapiens
tacctccctccctccatcc

SEQ ID NO: 203 - DNA, 24 bp, Homo sapiens
  misc_feature (1)..(1): May be labeled with a 5′ 6-FAM, VIC, NED, JOE, ROX, PET, TAMRA or 5-FAM fluorescent moiety
ggggaatacatttgtgtaggtagg

SEQ ID NO: 204 - DNA, 25 bp, Homo sapiens
cactaatacctggtgaatgattctt

SEQ ID NO: 205 - DNA, 25 bp, Homo sapiens
  misc_feature (1)..(1): May be labeled with a 5′ 6-FAM, VIC, NED, JOE, ROX, PET, TAMRA or 5-FAM fluorescent moiety
aaatgaagatattttcttagcttat

SEQ ID NO: 206 - DNA, 27 bp, Homo sapiens
gcattcaatattttacttttaagaatc

SEQ ID NO: 207 - DNA, 23 bp, Homo sapiens
  misc_feature (1)..(1): May be labeled with a 5′ 6-FAM, VIC, NED, JOE, ROX, PET, TAMRA or 5-FAM fluorescent moiety
gggtaaagaaaatgtgctctctc

SEQ ID NO: 208 - DNA, 21 bp, Homo sapiens
ggttctccaagttcaatggtg

SEQ ID NO: 209 - DNA, 21 bp, Homo sapiens
  misc_feature (1)..(1): May be labeled with a 5′ 6-FAM, VIC, NED, JOE, ROX, PET, TAMRA or 5-FAM fluorescent moiety
cagcagaaagggaatagttgg

SEQ ID NO: 210 - DNA, 24 bp, Homo sapiens
caagttaaaaacacaaaatggaaa

SEQ ID NO: 211 - DNA, 23 bp, Homo sapiens
  misc_feature (1)..(1): May be labeled with a 5′ 6-FAM, VIC, NED, JOE, ROX, PET, TAMRA or 5-FAM fluorescent moiety
tggatggatggatagatacacag

SEQ ID NO: 212 - DNA, 19 bp, Homo sapiens
cccactcccagccttctaa

SEQ ID NO: 213 - DNA, 24 bp, Homo sapiens
  misc_feature (1)..(1): May be labeled with a 5′ 6-FAM, VIC, NED, JOE, ROX, PET, TAMRA or 5-FAM fluorescent moiety
tctctgtgtctatgtgtgcatgtt

SEQ ID NO: 214 - DNA, 20 bp, Homo sapiens
ggttttgcaaaggcaggtta

SEQ ID NO: 215 - DNA, 27 bp, Homo sapiens
  misc_feature (1)..(1): May be labeled with a 5′ 6-FAM, VIC, NED, JOE, ROX, PET, TAMRA or 5-FAM fluorescent moiety
cactttcacaatatgtattgcttatca

SEQ ID NO: 216 - DNA, 24 bp, Homo sapiens
ggtagcaatttcactctctctttc

SEQ ID NO: 217 - DNA, 27 bp, Homo sapiens
  misc_feature (1)..(1): May be labeled with a 5′ 6-FAM, VIC, NED, JOE, ROX, PET, TAMRA or 5-FAM fluorescent moiety
gatgacaagacagattagatagattgg

SEQ ID NO: 218 - DNA, 24 bp, Homo sapiens
cattgggcttatttttctttctat

SEQ ID NO: 219 - DNA, 18 bp, Homo sapiens
  misc_feature (1)..(1): May be labeled with a 5′ 6-FAM, VIC, NED, JOE, ROX, PET, TAMRA or 5-FAM fluorescent moiety
aaaggcaggaaggcagga

SEQ ID NO: 220 - DNA, 25 bp, Homo sapiens
ttttcttctttttgcttctcttttc

SEQ ID NO: 221 - DNA, 25 bp, Homo sapiens
  misc_feature (1)..(1): May be labeled with a 5′ 6-FAM, VIC, NED, JOE, ROX, PET, TAMRA or 5-FAM fluorescent moiety
tgatgtagttcactaggatgtaggg

SEQ ID NO: 222 - DNA, 20 bp, Homo sapiens
cctgagacacaagagcgaga

SEQ ID NO: 223 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
cctgggtgacaagagtgaaa

SEQ ID NO: 224 - DNA, 24 bp, Homo sapiens
cacagaaatttgtagaaccacagc

SEQ ID NO: 225 - DNA, 21 bp, Homo sapiens
  misc_feature (1)..(1): May be labeled with a 5′ 6-FAM, VIC, NED, JOE, ROX, PET, TAMRA or 5-FAM fluorescent moiety
ggcatcaaaatttggaagaaa

SEQ ID NO: 226 - DNA, 20 bp, Homo sapiens
aaagctgagctgaatggtga

SEQ ID NO: 227 - DNA, 23 bp, Homo sapiens
  misc_feature (1)..(1): May be labeled with a 5′ 6-FAM, VIC, NED, JOE, ROX, PET, TAMRA or 5-FAM fluorescent moiety
gggaaaccacagttatacattcc

SEQ ID NO: 228 - DNA, 26 bp, Homo sapiens
tgtttacagttcttcacagagttctt

SEQ ID NO: 229 - DNA, 22 bp, Homo sapiens
  misc_feature (1)..(1): May be labeled with a 5′ 6-FAM, VIC, NED, JOE, ROX, PET, TAMRA or 5-FAM fluorescent moiety
accacagaattcagtccaaaaa

SEQ ID NO: 230 - DNA, 20 bp, Homo sapiens
ttatcccctggaggaacttg

SEQ ID NO: 231 - DNA, 22 bp, Homo sapiens
  misc_feature (1)..(1): May be labeled with a 5′ 6-FAM, VIC, NED, JOE, ROX, PET, TAMRA or 5-FAM fluorescent moiety
ccagaaaatgacacatgaagga

SEQ ID NO: 232 - DNA, 26 bp, Homo sapiens
gagatattggcctgtagttttctttt

SEQ ID NO: 233 - DNA, 26 bp, Homo sapiens
  misc_feature (1)..(1): May be labeled with a 5′ 6-FAM, VIC, NED, JOE, ROX, PET, TAMRA or 5-FAM fluorescent moiety
gatgttgtattagtcaatgttctcca

SEQ ID NO: 234 - DNA, 24 bp, Homo sapiens
tctgtctgtctgtctgtctgtctg

SEQ ID NO: 235 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
atgggcaaacagatgggtag

SEQ ID NO: 236 - DNA, 23 bp, Homo sapiens
ccatattatcatccatccatcca

SEQ ID NO: 237 - DNA, 22 bp, Homo sapiens
  misc_feature (1)..(1): May be labeled with a 5′ 6-FAM, VIC, NED, JOE, ROX, PET, TAMRA or 5-FAM fluorescent moiety
gggtaacagcataagaccctgt

SEQ ID NO: 238 - DNA, 20 bp, Homo sapiens
tcactttggtgcttgctttg

SEQ ID NO: 239 - DNA, 27 bp, Homo sapiens
  misc_feature (1)..(1): May be labeled with a 5′ 6-FAM, VIC, NED, JOE, ROX, PET, TAMRA or 5-FAM fluorescent moiety
gaatctgctgagagagtagattttaag

SEQ ID NO: 240 - DNA, 23 bp, Homo sapiens
tgcatcacctcacataggttact

SEQ ID NO: 241 - DNA, 22 bp, Homo sapiens
  misc_feature (1)..(1): May be labeled with a 5′ 6-FAM, VIC, NED, JOE, ROX, PET, TAMRA or 5-FAM fluorescent moiety
tgctgacaggctacagaacttt

SEQ ID NO: 242 - DNA, 27 bp, Homo sapiens
tctgcatttgtagaaataatcttatca

SEQ ID NO: 243 - DNA, 24 bp, Homo sapiens
  misc_feature (1)..(1): May be labeled with a 5′ 6-FAM, VIC, NED, JOE, ROX, PET, TAMRA or 5-FAM fluorescent moiety
accattattctcctgagtcctctc

SEQ ID NO: 244 - DNA, 24 bp, Homo sapiens
acattctagtgctacagggtattc

SEQ ID NO: 245 - DNA, 25 bp, Homo sapiens
  misc_feature (1)..(1): May be labeled with a 5′ 6-FAM, VIC, NED, JOE, ROX, PET, TAMRA or 5-FAM fluorescent moiety
gtcccactatctcaataatctgatg

SEQ ID NO: 246 - DNA, 20 bp, Homo sapiens
actggtcaccttcatcacca

SEQ ID NO: 247 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
ggagaaaggggaggtgtaga

SEQ ID NO: 248 - DNA, 21 bp, Homo sapiens
ccatcctcctcccttcttttt

SEQ ID NO: 249 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
ttgccatgtcaattgctaca

SEQ ID NO: 250 - DNA, 26 bp, Homo sapiens
tgtttcttgacttaacattttcttct

SEQ ID NO: 251 - DNA, 21 bp, Homo sapiens
  misc_feature (1)..(1): May be labeled with a 5′ 6-FAM, VIC, NED, JOE, ROX, PET, TAMRA or 5-FAM fluorescent moiety
gatttgaaaatgagcagtcca

SEQ ID NO: 252 - DNA, 23 bp, Homo sapiens
gcatgtttcaggtcttttattgc

SEQ ID NO: 253 - DNA, 27 bp, Homo sapiens
  misc_feature (1)..(1): May be labeled with a 5′ 6-FAM, VIC, NED, JOE, ROX, PET, TAMRA or 5-FAM fluorescent moiety
gcctgttagtttttattgttatcttag

SEQ ID NO: 254 - DNA, 24 bp, Homo sapiens
tttttatcagaagcatgaaaacag

SEQ ID NO: 255 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
ctgtttctcctccctgcaac

SEQ ID NO: 256 - DNA, 20 bp, Homo sapiens
ggagcgtatctgtgcgtgta

SEQ ID NO: 257 - DNA, 25 bp, Homo sapiens
  misc_feature (1)..(1): May be labeled with a 5′ 6-FAM, VIC, NED, JOE, ROX, PET, TAMRA or 5-FAM fluorescent moiety
cacttctaggtttttacccaagtga

SEQ ID NO: 258 - DNA, 27 bp, Homo sapiens
tgaagtaactctgaacactcaatactt

SEQ ID NO: 259 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
gtttaaccaggagcccttcc

SEQ ID NO: 260 - DNA, 25 bp, Homo sapiens
gagcaactacctactatggttcctt

SEQ ID NO: 261 - DNA, 19 bp, Homo sapiens
  misc_feature (1)..(1): May be labeled with a 5′ 6-FAM, VIC, NED, JOE, ROX, PET, TAMRA or 5-FAM fluorescent moiety
actccagcctgggcgatag

SEQ ID NO: 262 - DNA, 27 bp, Homo sapiens
cttggatttatgtatctctcctagagt

SEQ ID NO: 263 - DNA, 21 bp, Homo sapiens
  misc_feature (1)..(1): May be labeled with a 5′ 6-FAM, VIC, NED, JOE, ROX, PET, TAMRA or 5-FAM fluorescent moiety
gctgttcttctaagtgccaca

SEQ ID NO: 264 - DNA, 20 bp, Homo sapiens
ggggttgttgcgaagattag

SEQ ID NO: 265 - DNA, 22 bp, Homo sapiens
  misc_feature (1)..(1): May be labeled with a 5′ 6-FAM, VIC, NED, JOE, ROX, PET, TAMRA or 5-FAM fluorescent moiety
tggagataaacacataggcaca

SEQ ID NO: 266 - DNA, 20 bp, Homo sapiens
taaggcaggagtcatgtcca

SEQ ID NO: 267 - DNA, 23 bp, Homo sapiens
  misc_feature (1)..(1): May be labeled with a 5′ 6-FAM, VIC, NED, JOE, ROX, PET, TAMRA or 5-FAM fluorescent moiety
gccaattacattgcatattgcat

SEQ ID NO: 268 - DNA, 24 bp, Homo sapiens
caacaaagcaataaagagttttgc

SEQ ID NO: 269 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
atggagtgccactggaagaa

SEQ ID NO: 270 - DNA, 21 bp, Homo sapiens
ccagttgagtttggacctcag

SEQ ID NO: 271 - DNA, 19 bp, Homo sapiens
  misc_feature (1)..(1): May be labeled with a 5′ 6-FAM, VIC, NED, JOE, ROX, PET, TAMRA or 5-FAM fluorescent moiety
ggccaaaattagcgtgaca

SEQ ID NO: 272 - DNA, 20 bp, Homo sapiens
caactccaggccaaatcatc

SEQ ID NO: 273 - DNA, 22 bp, Homo sapiens
  misc_feature (1)..(1): May be labeled with a 5′ 6-FAM, VIC, NED, JOE, ROX, PET, TAMRA or 5-FAM fluorescent moiety
cggagtgaaagaagattgattt

SEQ ID NO: 274 - DNA, 24 bp, Homo sapiens
ttgacaatttagcagcatgtattt

SEQ ID NO: 275 - DNA, 19 bp, Homo sapiens
  misc_feature (1)..(1): May be labeled with a 5′ 6-FAM, VIC, NED, JOE, ROX, PET, TAMRA or 5-FAM fluorescent moiety
agcctcatgcctggggata

SEQ ID NO: 276 - DNA, 26 bp, Homo sapiens
attttcttcatttggtgttattttgg

SEQ ID NO: 277 - DNA, 26 bp, Homo sapiens
  misc_feature (1)..(1): May be labeled with a 5′ 6-FAM, VIC, NED, JOE, ROX, PET, TAMRA or 5-FAM fluorescent moiety
cttttcagattaaatgagacaatatg

SEQ ID NO: 278 - DNA, 25 bp, Homo sapiens
tgctaatgtgtgattatatgtacgc

SEQ ID NO: 279 - DNA, 21 bp, Homo sapiens
  misc_feature (1)..(1): May be labeled with a 5′ 6-FAM, VIC, NED, JOE, ROX, PET, TAMRA or 5-FAM fluorescent moiety
tgagtgacaaattgagacctt

SEQ ID NO: 280 - DNA, 26 bp, Homo sapiens
gtcttacaataacagttgctactatt

SEQ ID NO: 281 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
aacaggggcacaagacagat

SEQ ID NO: 282 - DNA, 20 bp, Homo sapiens
cccactcctgtgcactctct

SEQ ID NO: 283 - DNA, 25 bp, Homo sapiens
  misc_feature (1)..(1): May be labeled with a 5′ 6-FAM, VIC, NED, JOE, ROX, PET, TAMRA or 5-FAM fluorescent moiety
gttgacaatagcacacatacagtcc

SEQ ID NO: 284 - DNA, 18 bp, Homo sapiens
accaccacccacaccatc

SEQ ID NO: 285 - DNA, 21 bp, Homo sapiens
  misc_feature (1)..(1): May be labeled with a 5′ 6-FAM, VIC, NED, JOE, ROX, PET, TAMRA or 5-FAM fluorescent moiety
tcttccattcccatcatttca

SEQ ID NO: 286 - DNA, 24 bp, Homo sapiens
tctccaggaacattgttttacttt

SEQ ID NO: 287 - DNA, 23 bp, Homo sapiens
  misc_feature (1)..(1): May be labeled with a 5′ 6-FAM, VIC, NED, JOE, ROX, PET, TAMRA or 5-FAM fluorescent moiety
cagccttttccaaattttacatc

SEQ ID NO: 288 - DNA, 20 bp, Homo sapiens
tgccctaccagaccaatttt

SEQ ID NO: 289 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
tggggcatacatagtgcaaa

SEQ ID NO: 290 - DNA, 20 bp, Homo sapiens
aaataaccgctggctctgtg

SEQ ID NO: 291 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
gactttgtgccatttctcca

SEQ ID NO: 292 - DNA, 20 bp, Homo sapiens
aatcttcaggctgcttgcac

SEQ ID NO: 293 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
ggcacaggaaacgtgaattt

SEQ ID NO: 294 - DNA, 22 bp, Homo sapiens
gctgttttcttctgttgtctgg

SEQ ID NO: 295 - DNA, 26 bp, Homo sapiens
  misc_feature (1)..(1): May be labeled with a 5′ 6-FAM, VIC, NED, JOE, ROX, PET, TAMRA or 5-FAM fluorescent moiety
tttggatgtctttctttcttctatca

SEQ ID NO: 296 - DNA, 22 bp, Homo sapiens
cagtggaaaccaaactacaacg

SEQ ID NO: 297 - DNA, 24 bp, Homo sapiens
  misc_feature (1)..(1): May be labeled with a 5′ 6-FAM, VIC, NED, JOE, ROX, PET, TAMRA or 5-FAM fluorescent moiety
caataaagacctgacttggaaaaa

SEQ ID NO: 298 - DNA, 25 bp, Homo sapiens
ctcaacacagcaacaaaaatataaa

SEQ ID NO: 299 - DNA, 25 bp, Homo sapiens
  misc_feature (1)..(1): May be labeled with a 5′ 6-FAM, VIC, NED, JOE, ROX, PET, TAMRA or 5-FAM fluorescent moiety
aagagaagaacatcactaagaacca

SEQ ID NO: 300 - DNA, 24 bp, Homo sapiens
aactcagtgttccacagtaactca

SEQ ID NO: 301 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
ctgaaggacctgcctgagat

SEQ ID NO: 302 - DNA, 25 bp, Homo sapiens
tactttttattgttagggtgtgctc

SEQ ID NO: 303 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
cccctgcataaactcactca

SEQ ID NO: 304 - DNA, 24 bp, Homo sapiens
ttccaaaggacataatccatattt

SEQ ID NO: 305 - DNA, 22 bp, Homo sapiens
  misc_feature (1)..(1): May be labeled with a 5′ 6-FAM, VIC, NED, JOE, ROX, PET, TAMRA or 5-FAM fluorescent moiety
ggacctaggttccaatttctcc

SEQ ID NO: 306 - DNA, 21 bp, Homo sapiens
tgtatggtgcatgaacctgtg

SEQ ID NO: 307 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
ctggcctccacccactagat

SEQ ID NO: 308 - DNA, 22 bp, Homo sapiens
ctttcaatgtcagaaggcattt

SEQ ID NO: 309 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
accctggagagtgactgcat

SEQ ID NO: 310 - DNA, 20 bp, Homo sapiens
cgcctgtactgcctgagttt

SEQ ID NO: 311 - DNA, 16 bp, Homo sapiens
  misc_feature (1)..(1): May be labeled with a 5′ 6-FAM, VIC, NED, JOE, ROX, PET, TAMRA or 5-FAM fluorescent moiety
ggctgcacaggcattc

SEQ ID NO: 312 - DNA, 20 bp, Homo sapiens
gggaatgcagtgaaatggac

SEQ ID NO: 313 - DNA, 21 bp, Homo sapiens
  misc_feature (1)..(1): May be labeled with a 5′ 6-FAM, VIC, NED, JOE, ROX, PET, TAMRA or 5-FAM fluorescent moiety
ttttgccacaaaaattaccaa

SEQ ID NO: 314 - DNA, 20 bp, Homo sapiens
aaatcaggaaatcggcactg

SEQ ID NO: 315 - DNA, 21 bp, Homo sapiens
  misc_feature (1)..(1): May be labeled with a 5′ 6-FAM, VIC, NED, JOE, ROX, PET, TAMRA or 5-FAM fluorescent moiety
tcagcacaatgctactgggta

SEQ ID NO: 316 - DNA, 20 bp, Homo sapiens
gactgggaacatggctcttc

SEQ ID NO: 317 - DNA, 25 bp, Homo sapiens
  misc_feature (1)..(1): May be labeled with a 5′ 6-FAM, VIC, NED, JOE, ROX, PET, TAMRA or 5-FAM fluorescent moiety
tgtggaaagttgtagataggatgaa

SEQ ID NO: 318 - DNA, 27 bp, Homo sapiens
tgaggatcacactttgagtagtaagtc

SEQ ID NO: 319 - DNA, 23 bp, Homo sapiens
  misc_feature (1)..(1): May be labeled with a 5′ 6-FAM, VIC, NED, JOE, ROX, PET, TAMRA or 5-FAM fluorescent moiety
ccaaactacattcttcttcctga

SEQ ID NO: 320 - DNA, 26 bp, Homo sapiens
gaggaatttatgctaagatttgaagg

SEQ ID NO: 321 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
aacacgcttgtcaaatgctt

SEQ ID NO: 322 - DNA, 27 bp, Homo sapiens
ttaagttattgttgttgtttcttgtgg

SEQ ID NO: 323 - DNA, 22 bp, Homo sapiens
  misc_feature (1)..(1): May be labeled with a 5′ 6-FAM, VIC, NED, JOE, ROX, PET, TAMRA or 5-FAM fluorescent moiety
cagaagcagatttgaacattgg

SEQ ID NO: 324 - DNA, 23 bp, Homo sapiens
gctataaacattcaccgttaggg

SEQ ID NO: 325 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
ggcctctctcccagaaagat

SEQ ID NO: 326 - DNA, 27 bp, Homo sapiens
tgtcaagaagtacctaccatattttga

SEQ ID NO: 327 - DNA, 21 bp, Homo sapiens
  misc_feature (1)..(1): May be labeled with a 5′ 6-FAM, VIC, NED, JOE, ROX, PET, TAMRA or 5-FAM fluorescent moiety
ctccctcttaatgttgcctgt

SEQ ID NO: 328 - DNA, 20 bp, Homo sapiens
tgagagtctttggtgggaga

SEQ ID NO: 329 - DNA, 22 bp, Homo sapiens
  misc_feature (1)..(1): May be labeled with a 5′ 6-FAM, VIC, NED, JOE, ROX, PET, TAMRA or 5-FAM fluorescent moiety
tgcttttggtgtcttcaatctg

SEQ ID NO: 330 - DNA, 21 bp, Homo sapiens
tggtcatgtaacagtgcttgg

SEQ ID NO: 331 - DNA, 23 bp, Homo sapiens
  misc_feature (1)..(1): May be labeled with a 5′ 6-FAM, VIC, NED, JOE, ROX, PET, TAMRA or 5-FAM fluorescent moiety
tgacatttaatcaaccaagaaat

SEQ ID NO: 332 - DNA, 22 bp, Homo sapiens
tttttgaactaagaacctggag

SEQ ID NO: 333 - DNA, 24 bp, Homo sapiens
  misc_feature (1)..(1): May be labeled with a 5′ 6-FAM, VIC, NED, JOE, ROX, PET, TAMRA or 5-FAM fluorescent moiety
ttgttagtgtttgcaaagtgatga

SEQ ID NO: 334 - DNA, 24 bp, Homo sapiens
ggatttatagatatgggagggttc

SEQ ID NO: 335 - DNA, 19 bp, Homo sapiens
  misc_feature (1)..(1): May be labeled with a 5′ 6-FAM, VIC, NED, JOE, ROX, PET, TAMRA or 5-FAM fluorescent moiety
cccttgatgtaggcacagg

SEQ ID NO: 336 - DNA, 20 bp, Homo sapiens
cgtgggggagtagtagtggt

SEQ ID NO: 337 - DNA, 19 bp, Homo sapiens
  misc_feature (1)..(1): May be labeled with a 5′ 6-FAM, VIC, NED, JOE, ROX, PET, TAMRA or 5-FAM fluorescent moiety
cttcttgcattccccatgc

SEQ ID NO: 338 - DNA, 20 bp, Homo sapiens
tttcctcacagcaaaagagg

SEQ ID NO: 339 - DNA, 19 bp, Homo sapiens
  misc_feature (1)..(1): May be labeled with a 5′ 6-FAM, VIC, NED, JOE, ROX, PET, TAMRA or 5-FAM fluorescent moiety
cctgggcaacaagagtgaa

SEQ ID NO: 340 - DNA, 27 bp, Homo sapiens
ggaaagagagttatatttaagagcaga

SEQ ID NO: 341 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
cccatctgtcctcctggata

SEQ ID NO: 342 - DNA, 21 bp, Homo sapiens
ggtcccctatttgtcttgtcc

SEQ ID NO: 343 - DNA, 24 bp, Homo sapiens
  misc_feature (1)..(1): May be labeled with a 5′ 6-FAM, VIC, NED, JOE, ROX, PET, TAMRA or 5-FAM fluorescent moiety
tccctttctctctctctctctctc

SEQ ID NO: 344 - DNA, 23 bp, Homo sapiens
tgatgtgttatgaattggcaaaa

SEQ ID NO: 345 - DNA, 22 bp, Homo sapiens
  misc_feature (1)..(1): May be labeled with a 5′ 6-FAM, VIC, NED, JOE, ROX, PET, TAMRA or 5-FAM fluorescent moiety
ggttggaatgactgaaggctta

SEQ ID NO: 346 - DNA, 23 bp, Homo sapiens
aagatagcaggcaacaataagat

SEQ ID NO: 347 - DNA, 21 bp, Homo sapiens
  misc_feature (1)..(1): May be labeled with a 5′ 6-FAM, VIC, NED, JOE, ROX, PET, TAMRA or 5-FAM fluorescent moiety
ggttctagggacactcccttc

SEQ ID NO: 348 - DNA, 19 bp, Homo sapiens
agaccatggcctgctttta

SEQ ID NO: 349 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
gccttacacacaagcacacc

SEQ ID NO: 350 - DNA, 20 bp, Homo sapiens
gcaccaatatcaaagcagca

SEQ ID NO: 351 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
accactcagccagtttgctt

SEQ ID NO: 352 - DNA, 19 bp, Homo sapiens
gaactggccttgccttcac

SEQ ID NO: 353 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
gggcaacaagagcaaaactc

SEQ ID NO: 354 - DNA, 20 bp, Homo sapiens
ccctgttggtaaatccttgg

SEQ ID NO: 355 - DNA, 25 bp, Homo sapiens
  misc_feature (1)..(1): May be labeled with a 5′ 6-FAM, VIC, NED, JOE, ROX, PET, TAMRA or 5-FAM fluorescent moiety
caaggaactcaaacaacagtttaca

SEQ ID NO: 356 - DNA, 21 bp, Homo sapiens
tctttgcccactttttaatgg

SEQ ID NO: 357 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
ggttctccagagggacagaa

SEQ ID NO: 358 - DNA, 24 bp, Homo sapiens
tctccctgataaactccttttcat

SEQ ID NO: 359 - DNA, 21 bp, Homo sapiens
  misc_feature (1)..(1): May be labeled with a 5′ 6-FAM, VIC, NED, JOE, ROX, PET, TAMRA or 5-FAM fluorescent moiety
tctctttcagtgacccctcct

SEQ ID NO: 360 - DNA, 21 bp, Homo sapiens
ggggaaagagagagagaggaa

SEQ ID NO: 361 - DNA, 24 bp, Homo sapiens
  misc_feature (1)..(1): May be labeled with a 5′ 6-FAM, VIC, NED, JOE, ROX, PET, TAMRA or 5-FAM fluorescent moiety
ccacaagcagataaagaaaatgtg

SEQ ID NO: 362 - DNA, 23 bp, Homo sapiens
tcatttttatggccatggtatgt

SEQ ID NO: 363 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
tatttcccccagcacccttt

SEQ ID NO: 364 - DNA, 27 bp, Homo sapiens
tttcagtaaaatcatacacctttaaca

SEQ ID NO: 365 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
atctttggaggggaaggagt

SEQ ID NO: 366 - DNA, 24 bp, Homo sapiens
gaagtatcgtatctgaatcccgta

SEQ ID NO: 367 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
ttcaaagagccctctgctgt

SEQ ID NO: 368 - DNA, 25 bp, Homo sapiens
acaattctgtatagactttgtgtgt

SEQ ID NO: 369 - DNA, 23 bp, Homo sapiens
  misc_feature (1)..(1): May be labeled with a 5′ 6-FAM, VIC, NED, JOE, ROX, PET, TAMRA or 5-FAM fluorescent moiety
tgagaactccctaaacaaaatgt

SEQ ID NO: 370 - DNA, 23 bp, Homo sapiens
ttccttgaatgtaaggattaggg

SEQ ID NO: 371 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
tttgcctaccacggttgtct

SEQ ID NO: 372 - DNA, 18 bp, Homo sapiens
acccgtcgtggttgtgat

SEQ ID NO: 373 - DNA, 23 bp, Homo sapiens
  misc_feature (1)..(1): May be labeled with a 5′ 6-FAM, VIC, NED, JOE, ROX, PET, TAMRA or 5-FAM fluorescent moiety
tcctgttttccagtaccagaagt

SEQ ID NO: 374 - DNA, 24 bp, Homo sapiens
gagtctattaggagcacaaaaagg

SEQ ID NO: 375 - DNA, 22 bp, Homo sapiens
  misc_feature (1)..(1): May be labeled with a 5′ 6-FAM, VIC, NED, JOE, ROX, PET, TAMRA or 5-FAM fluorescent moiety
ttgactagaaatgctccctcaa

SEQ ID NO: 376 - DNA, 21 bp, Homo sapiens
caggtttctgtgtggacattg

SEQ ID NO: 377 - DNA, 19 bp, Homo sapiens
  misc_feature (1)..(1): May be labeled with a 5′ 6-FAM, VIC, NED, JOE, ROX, PET, TAMRA or 5-FAM fluorescent moiety
actccagcctgagcaacag

SEQ ID NO: 378 - DNA, 19 bp, Homo sapiens
cattgctcccccaacaact

SEQ ID NO: 379 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
gagggcaacagagtggagac

SEQ ID NO: 380 - DNA, 26 bp, Homo sapiens
tgattttgtacacatttatggggtat

SEQ ID NO: 381 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
ccttcacatgtatccccaaa

SEQ ID NO: 382 - DNA, 23 bp, Homo sapiens
tgaatggataaagaaaatgtggt

SEQ ID NO: 383 - DNA, 22 bp, Homo sapiens
  misc_feature (1)..(1): May be labeled with a 5′ 6-FAM, VIC, NED, JOE, ROX, PET, TAMRA or 5-FAM fluorescent moiety
aaactgtggaaattgcttccat

SEQ ID NO: 384 - DNA, 23 bp, Homo sapiens
tcaacaaattccaggttatgtca

SEQ ID NO: 385 - DNA, 24 bp, Homo sapiens
  misc_feature (1)..(1): May be labeled with a 5′ 6-FAM, VIC, NED, JOE, ROX, PET, TAMRA or 5-FAM fluorescent moiety
ttttaaaaacttctacaatgagca

SEQ ID NO: 386 - DNA, 26 bp, Homo sapiens
cctaggtaacatagtgagaccttgtc

SEQ ID NO: 387 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
atgatgcctggcacacagta

SEQ ID NO: 388 - DNA, 22 bp, Homo sapiens
aagcactttgaatcatttacgg

SEQ ID NO: 389 - DNA, 20 bp, Homo sapiens
  misc_feature (1)..(1): May be labeled with a 5′ 6-FAM, VIC, NED, JOE, ROX, PET, TAMRA or 5-FAM fluorescent moiety
acccccacctccctgaaata

SEQ ID NO: 390 - DNA, 20 bp, Homo sapiens
tgccctactccattccacac

SEQ ID NO: 391 - DNA, 22 bp, Homo sapiens
  misc_feature (1)..(1): May be labeled with a 5′ 6-FAM, VIC, NED, JOE, ROX, PET, TAMRA or 5-FAM fluorescent moiety
ccacttgtcctcttgctacaca

SEQ ID NO: 392 - DNA, 24 bp, Homo sapiens
tggcttagagtactttttcactgc

SEQ ID NO: 393 - DNA, 22 bp, Homo sapiens
  misc_feature (1)..(1): May be labeled with a 5′ 6-FAM, VIC, NED, JOE, ROX, PET, TAMRA or 5-FAM fluorescent moiety
tccaaaataacactgaaacacg

SEQ ID NO: 394 - DNA, 20 bp, Homo sapiens
aagggtttactcccccaaaa

SEQ ID NO: 395 - DNA, 24 bp, Homo sapiens
  misc_feature (1)..(1): May be labeled with a 5′ 6-FAM, VIC, NED, JOE, ROX, PET, TAMRA or 5-FAM fluorescent moiety
ggtataaaactgaactcatcagca

SEQ ID NO: 396 - DNA, 25 bp, Homo sapiens
agctgtagagttgaggaatgttttc

SEQ ID NO: 397 - DNA, 26 bp, Homo sapiens
  misc_feature (1)..(1): May be labeled with a 5′ 6-FAM, VIC, NED, JOE, ROX, PET, TAMRA or 5-FAM fluorescent moiety
aaacatttggttaggctaatttctat

SEQ ID NO: 398 - DNA, 23 bp, Homo sapiens
aaacaaatgcgaatttaaaaaga

SEQ ID NO: 399 - DNA, 22 bp, Homo sapiens
  misc_feature (1)..(1): May be labeled with a 5′ 6-FAM, VIC, NED, JOE, ROX, PET, TAMRA or 5-FAM fluorescent moiety
ggagatttcttccttgttgcac

SEQ ID NO: 400 - DNA, 21 bp, Homo sapiens
gctaggctgtgtgtgtctgtg

SEQ ID NO: 401 - DNA, 22 bp, Homo sapiens
  misc_feature (1)..(1): May be labeled with a 5′ 6-FAM, VIC, NED, JOE, ROX, PET, TAMRA or 5-FAM fluorescent moiety
aaaggcaaagaaaaactgttgc

SEQ ID NO: 402 - DNA, 27 bp, Homo sapiens
gatcattcatataacctcaaaagaact

SEQ ID NO: 403 - DNA, 21 bp, Homo sapiens
  misc_feature (1)..(1): May be labeled with a 5′ 6-FAM, VIC, NED, JOE, ROX, PET, TAMRA or 5-FAM fluorescent moiety
ggcatcgtattcattgttcca

SEQ ID NO: 404 - DNA, 24 bp, Homo sapiens
aggttcttccaaattatttttgtg

SEQ ID NO: 405 - DNA, 19 bp, Homo sapiens
  misc_feature (1)..(1): May be labeled with a 5′ 6-FAM, VIC, NED, JOE, ROX, PET, TAMRA or 5-FAM fluorescent moiety
tgaaacactgctccccttg

SEQ ID NO: 406 - DNA, 23 bp, Homo sapiens
ccgagttattacaaagaagcaca

(19) United States
(12) Patent Application Publication
(10) Pub. No.: US 2010/0261188 A1
(43) Pub. Date: Oct. 14, 2010

(54) NON-INVASIVE PRENATAL GENETIC SCREEN

(75) Inventors: Ram Bhatt, San Diego, CA (US); Wen-Hua Fan, San Diego, CA (US); Roger Tim, La Jolla, CA (US); Farideh Z. Bischoff, Sugar Land, TX (US)

Correspondence Address: COOLEY LLP;ATTN: Patent Group, Suite 1100, 777 - 6th Street, NW, WASHINGTON, DC, 20001, US

(73) Assignee: BIOCEPT, INC., San Diego, CA (US)

(21) Appl. No.: 12/768,388

(22) Filed: Apr. 27, 2010

Related U.S. Application Data
(63) Continuation of application No. 11/952,459, filed on Dec. 7, 2007, now abandoned.
(60) Provisional application No. 60/869,090, filed on Dec. 7, 2006.

(51) Int. Cl. C12Q 1/68; C07H 21/04

(52) U.S. Cl. 435/6; 536/23.1

(57) ABSTRACT

The present invention provides methods and kits useful for genetic testing or screening of fetuses using nucleic acid samples isolated from cervical mucus samples of fetus hosts.

### FIELD OF THE INVENTION

This invention relates generally to the isolation of fetal nucleic acid and prenatal screening or testing of genetic and chromosomal abnormalities.

### BACKGROUND OF THE INVENTION

Prenatal testing or screening is usually performed to determine the gender of the fetus or to detect genetic disorders and/or chromosomal abnormalities in the fetus during pregnancy. As of today, over 4000 genetic disorders, caused by one or more faulty genes, have been recognized. Some examples include Cystic Fibrosis, Huntington's Disease, Beta Thalassaemia, Myotonic Dystrophy, Sickle Cell Anemia, Porphyria, and Fragile-X-Syndrome. Chromosomal abnormality is caused by aberrations in chromosome numbers, duplication or absence of chromosomal material, and by defects in chromosome structure. Some examples of chromosomal abnormalities are trisomies, namely trisomy 16, a major cause of miscarriage in the first trimester, trisomy 21 (Down syndrome), trisomy 13 (Patau syndrome), trisomy 18 (Edwards syndrome), Klinefelter's syndrome (47, XXY), (47, XYY), and (47, XXX); the absence of chromosomes (monosomy), e.g., Turner syndrome (45, X0); chromosomal translocations, deletions and/or microdeletions, e.g., Robertsonian translocation, Angelman syndrome, DiGeorge syndrome and Wolf-Hirschhorn Syndrome.

Currently available prenatal genetic tests usually involve invasive procedures. For example, chorionic villous sampling (CVS) performed on a pregnant woman around 10-12 weeks into the pregnancy and amniocentesis performed at around 14-16 weeks all contain invasive procedures to obtain the sample for testing chromosomal abnormalities in a fetus. Fetal cells obtained via these sampling procedures are usually tested for chromosomal abnormalities using cytogenetic or fluorescent in situ hybridization (FISH) analyses.

While these procedures can be useful for detecting chromosomal aberrations, they have been shown to be associated with the risk of miscarriage. Therefore amniocentesis or CVS is only offered to women perceived to be at increased risk, including those of advanced maternal age (>35 years), those with abnormal maternal serum screening or those who have had a previous fetal chromosomal abnormality. As a result of these tests the percentage of women over the age of 35 who give birth to babies with chromosomal aberrations such as Down syndrome has drastically reduced. However, lack of appropriate or relatively safe prenatal testing or screening for the majority of pregnant women has resulted in about 80% of Down syndrome babies born to women under 35 years of age.

Thus there is a need for diagnostic screening tests for the general population of pregnant women, especially tests directed to identifying fetal chromosomal aberrations as well as other genetic variations or defects.

### SUMMARY OF THE INVENTION

The present invention is based, in part, on the discovery that cervical mucous is a good natural reservoir for migrated placental cells, e.g., fetal cells as well as for isolating fetal nucleic acids. Accordingly the present invention provides methods and kits useful for testing or screening for genetic abnormalities in fetuses using fetal nucleic acids isolated from cervical mucus samples. In addition, the present invention provides primers and probes useful for nucleic acid amplification of, e.g., genetic markers, especially using relatively small size amplicons in fetal genetic screening.

In one embodiment of the invention, it provides a method for conducting a genetic test of a fetus. The method comprises isolating a nucleic acid sample from a cervical mucus sample obtained from a female subject containing the fetus, wherein the nucleic acid sample consists essentially of polynucleotides in a size ranging from about 50 base pairs to about 300 base pairs and wherein the result of a genetic test on the nucleic acid sample is indicative of a genetic composition of the fetus.

In another embodiment of the invention, it provides a method of isolating a fetal nucleic acid sample. The method comprises isolating a nucleic acid sample consisting essentially of polynucleotides of about 50 base pairs to about 300 base pairs in length from a cervical mucus sample obtained from a female subject containing the fetus.

In yet another embodiment of the invention, it provides a genetic-testing kit suitable for testing the genetic composition of a fetus. The kit comprises a pair of primers suitable for amplifying a desired allele or genetic marker, wherein the amplified nucleotide fragment is less than about 200 base pairs and wherein the desired allele is not uniquely associated with the Y chromosome. In other embodiments, the kit comprises an isolated DNA sample from a cervical mucus sample obtained from a female subject containing the fetus. The DNA sample consists essentially of polynucleotides in a size ranging from about 50 base pairs to about 200 base pairs.

In still another embodiment of the invention, it provides an isolated DNA sample useful for genetic testing of a fetus. The DNA sample can be obtained by isolating DNA fragments in a size ranging from about 50 base pairs to about 200 base pairs from a cervical mucus sample obtained from a female subject containing the fetus.

### BRIEF DESCRIPTION OF THE FIGURES

FIG. 1 shows the size fractionation of total DNA obtained from cervical mucus on a 10% polyacrylamide gel. “Band A,” corresponding to a polynucleotide length of around 50-200 base pairs contains fetal DNA.

FIG. 2 shows an example of PCR electropherogram demonstrating that in one experiment the fetal signals match between fetal tissue DNA and the 50-200 by fragment of DNA isolated from a cervical mucus sample.

FIG. 3 shows another example of PCR electropherogram demonstrating that in another experiment the fetal signals match between fetal tissue DNA and the 50-200 by fragment of DNA isolated from a cervical mucus sample.

### DETAILED DESCRIPTION OF THE INVENTION

It is the discovery of the present invention that cervical mucus samples can be a great source for fetal cells as well as fetal nucleic acids. Accordingly, the present invention provides methods, reagents and kits useful for testing or screening fetus for genetic abnormalities using nucleic acids isolated from cervical mucus samples.

In addition, the present invention provides primers and probes useful for nucleic acid amplification, e.g., of genetic markers, especially using relatively small size amplicons in fetal genetic screening.

According to one aspect of the present invention, it provides methods for conducting genetic tests of a fetus by isolating one or more nucleic acid samples from one or more cervical mucus samples obtained from a female subject containing the fetus. In general, the nucleic acid sample useful for the methods of the present invention can be a DNA sample, RNA sample, or a combination thereof including any DNA, cDNA, or RNA derived from one or more nucleic acid samples isolated from one or more cervical mucus samples.

In one embodiment, the nucleic acid sample useful for the methods of the present invention is a DNA sample. In another embodiment, the nucleic acid sample useful for the methods of the present invention is substantially free of proteins or polypeptides. In yet another embodiment, the nucleic acid sample useful for the methods of the present invention is isolated by any known or later discovered size fractionation method including, but not limited to, gel electrophoresis, capillary electrophoresis, size exclusion matrixes, and size fractionation columns.

In still another embodiment, the nucleic acid sample useful for the methods of the present invention is in a size range representative of, or substantially associated, with fetal nucleic acid. In still another embodiment, the nucleic acid sample useful for the methods of the present invention is in a size range substantially free of nucleic acid from the host of the fetus. For example, the nucleic acid sample useful for the methods of the present invention can be in a size range from about 50 to about 1000 base pairs, from about 50 to about 500 base pairs, from about 50 to about 400 base pairs, from about 50 to about 300 base pairs, from about 50 to about 250 base pairs, from about 50 to about 200 base pairs, from about 50 to about 150 base pairs, or from about 50 to about 100 base pairs or a combination thereof, and optionally, does not contain a substantial amount, e.g., more than 0.5%, 1%, 2%, 3%, 4%, or 5% of nucleic acids from any other size range or source.

According to the present invention, the nucleic acid sample useful for the methods of the present invention can be isolated from a cervical mucus sample from the host of a fetus, e.g., a pregnant woman. The cervical mucus sample of the present invention can be obtained from the host of a fetus, at any time during the pregnancy, for example, during the first or second trimester, by any means now known or later discovered in the art. In general, a cervical mucus sample, e.g., an endocervical mucus sample, can be obtained using techniques such as transcervical swabs, endocervical lavage, scrapes, cytobrush, aspiration, intrauterine lavage, or a combination thereof.

In one embodiment, the cervical mucus sample of the present invention is a fresh sample, e.g., without substantial preservation or processing. In another embodiment, the cervical mucus sample is a sample preserved from a fresh sample, e.g., preserved in a suitable aqueous preservation or transportation medium, or alternatively, a sample of a medium containing nucleic acids leached from one or more cervical mucus samples. Without being bound to any theory, it is believed that nucleic acid will diffuse out from the cervical mucus into a fluid that is in contact with the mucus. Fetal nucleic acid will thus be present both in the cervical mucus sample as well as in the media in which the sample is stored and/or transported. Accordingly, the nucleic acid sample useful for the methods of the present invention can be obtained directly from the cervical mucus sample, or from the medium, for example, preservation medium, transportation medium, or any aqueous medium, that is in contact with the cervical mucus. Examples of transportation media include, but are not limited to, any tissue culture medium known to one of skill in the art, e.g., RPMI-1640 medium. In yet another embodiment, the cervical mucus sample of the present invention is maintained or stored between about 4° C. and about 20° C., e.g., in a low calcium basal medium.

In still another embodiment, the cervical mucus sample of the present invention is a treated sample, e.g., a fresh sample or preserved sample treated with any suitable reagent(s) to facilitate mucous dissolution which in turn, assists in isolation of nucleic acid components from the sample. For example, the cervical mucus sample can be a sample treated with mucolytic agent(s) or mucinase(s), e.g., N-acetyl-L-cysteine, L-cysteine, dithiothreitol (DTT), bromhexine hydrochloride, and any of the hyaluronidases, including hyaluronate lyase, hyaluronoglucosaminidase, and hyaluronglucuronidase. In another example, the cervical mucus sample of the present invention is a sample treated with enzyme(s), e.g., sugar hydrolysis enzyme(s) such as β-galactosidase or invertase, or proteinase, or pepsin or combinations thereof. The cervical mucus sample may also be treated with chemicals known in the art to induce apoptosis to release fetal nucleic acid.

In another embodiment, the cervical mucus sample of the present invention is a sample treated to enrich fetal nucleic acid and/or reduce maternal nucleic acid content. For example, the cervical mucus sample can be treated to reduce or degrade any nucleic acid, e.g. DNA that is characteristic of maternal DNA. One of such nucleic acid is hypermethylated maternal DNA. Any means to reduce, degrade, or selectively remove hypermethylated maternal DNA can be used including, without any limitation, methylation specific restriction enzymes such as McrBC (BioLabs), antibodies specific for hypermethylated maternal DNA such as anti-5′-methyl-cytosine antibodies and/or anti-methylCpG binding protein-2 (MeCP2) antibodies, or ligands or proteins such as MeCP2 that specifically bind methylated CpG islands in maternal DNA.

Alternatively fetal nucleic acid can be enriched using markers specific for fetal nucleic acids. For example, hypomethylated maspin DNA can be used as a marker for fetal DNA. In one instance, one can treat total cervical mucous DNA with sodium bisulfite, which can induce chemical changes in the hypomethylated fetal DNA whereby unmethylated cytosine of fetal DNA is converted into uridine (U). Such change can be used to preferentially isolate or enrich fetal DNA, e.g., to preferentially amplify fetal DNA containing uridine(s) converted from cytosine(s).

According to the present invention, the nucleic acid sample of the present invention can be used to conduct genetic tests or screening of a fetus. In particular, the nucleic acid sample of the present invention can be used to test or screen the genetic composition of a fetus, e.g., chromosomal composition, gene composition, or genetic marker or finger printing pattern of a fetus. In one embodiment, testing or screening a genetic composition of a fetus includes probing for chromosomal abnormalities including, without any limitation, monosomy, partial monosomy, trisomy, partial trisomy, chromosomal translocation, chromosomal duplication, chromosomal deletion or microdeletion, and chromosomal inversion.

In general, the term “monosomy” refers to the presence of only one chromosome from a pair of chromosomes. Monosomy is a type of aneuploidy. Partial monosomy occurs when the long or short arm of a chromosome is missing. Common human genetic disorders arising from monosomy include: X0, only one X chromosome instead of the usual two (XX) seen in a normal female (also known as Turner syndrome); cri du chat syndrome, a partial monosomy caused by a deletion of the end of the short p (from the word petit, French for small) arm of chromosome 5; and 1p36 Deletion Syndrome, a partial monosomy caused by a deletion at the end of the short p arm of chromosome 1.

In contrast, the term “trisomy” refers to the presence of three, instead of the normal two, chromosomes of a particular numbered type in an organism. Thus the presence of an extra chromosome 21 is called trisomy 21. Most trisomies, like most other abnormalities in chromosome number, result in distinctive birth defects. Many trisomies result in miscarriage or death at an early age. A partial trisomy occurs when part of an extra chromosome is attached to one of the other chromosomes, or if one of the chromosomes has two copies of part of its chromosome. A mosaic trisomy is a condition where extra chromosomal material exists in only some of the organism's cells. While a trisomy can occur with any chromosome, few babies survive to birth with most trisomies. The most common types that survive without spontaneous abortion in humans include: Trisomy 21 (Down syndrome); Trisomy 18 (Edwards syndrome); Trisomy 13 (Patau syndrome); Trisomy 9; Trisomy 8 (Warkany syndrome 2); Trisomy 16 (which is the most common trisomy in humans, occurring in more than 1% of pregnancies. This condition, however, usually results in spontaneous miscarriage in the first trimester). Trisomy involving sex chromosomes include: XXX (Triple X syndrome); XXY (Klinefelter's syndrome); and XYY (XYY syndrome).

In another embodiment, testing or screening a genetic composition of a fetus includes probing for allele or gene abnormalities, e.g., one or more mutations such as point mutations, insertions, deletions in one or more genes.

In yet another embodiment, testing or screening a genetic composition of a fetus includes probing for one or more polymorphism patterns or genetic markers, e.g., short tandem repeat sequences (STRs), single nucleotide polymorphisms (SNPs), etc.

In still another embodiment, testing or screening a genetic composition of a fetus includes probing for any genetic abnormality corresponding to or associated with a condition or disorder, e.g., Cystic Fibrosis, Sickle-Cell Anemia, Phenylketonuria, Tay-Scahs Disease, Adrenal Hyperplasia, Fanconi Anemia, Spinal Muscularatrophy, Duchenne's Muscular Dystrophy, Huntington's Disease, Beta Thalassaemia, Myotonic Dystrophy, Fragile-X Syndrome, Down Syndrome, Edwards Syndrome, Patau. Syndrome, Klinefelter's Syndrome, Triple X syndrome, XYY syndrome, Trisomy 8, Trisomy 16, Turner Syndrome, Robertsonian translocation, Angelman syndrome, DiGeorge Syndrome, Wolf-Hirschhorn Syndrome, RhD Syndrome, Tuberous Sclerosis, Ataxia Telangieltasia, and Prader-Willi syndrome.

In still another embodiment, testing or screening a genetic composition of a fetus includes probing for any genetic abnormality that is not uniquely associated with Y chromosome.

In still another embodiment, testing or screening a genetic composition of a fetus includes probing for any genetic condition corresponding to or associated with gender or paternity of the fetus.

Usually genetic tests provided by the present invention use the nucleic acid sample of the present invention either directly or as templates for “amplification-based” genetic composition testing assays, including without any limitation, polymerase chain reaction (“PCR”), real-time polymerase chain reaction (“RT-PCR”), ligase chain reaction (“LCR”), self-sustained sequence replication (“3SR”) also known as nucleic acid sequence based amplification (“NASBA”), Q-B-Replicase amplification, rolling circle amplification (“RCA”), transcription mediated amplification (“TMA”), linker-aided DNA amplification (“LADA”), multiple displacement amplification (“MDA”), invader and strand displacement amplification (“SDA”). Amplification of a nucleotide fragment using a pair of primers specific for an allele indicates the presence of the allele.

In one embodiment, the “amplification-based” genetic composition testing assays of the present invention include using primers to generate amplicons less than about 200 base pairs, less than about 150 base pairs, or between about 75 to about 150 base pairs. Exemplary primers of the invention used in the amplification-based assays are provided herein. In one embodiment, the primers of the invention include, but are not limited to, the pairs of primers of SEQ ID NOs: 1 and 2; SEQ ID NOs: 3 and 4; SEQ ID NOs: 5 and 6; SEQ ID NOs: 9 and 10; SEQ ID NOs: 11 and 12; and SEQ ID NOs: 13 and 14. In another embodiment, exemplary primers of the invention include, but are not limited to, the primer sets listed in Tables 2, 3, 4 and 5.

According to another aspect of the present invention, it provides a method of isolating a fetal nucleic acid sample. The method comprises isolating one or more nucleic acid samples from a cervical mucus sample obtained from a maternal host of a fetus in a size range enriched with fetal nucleic acids. Examples of such size range include without any limitation from about 50 to about 1000 base pairs, from about 50 to about 500 base pairs, from about 50 to about 400 base pairs, from about 50 to about 300 base pairs, from about 50 to about 250 base pairs, from about 50 to about 200 base pairs, from about 50 to about 150 base pairs, or from about 50 to about 100 base pairs or a combination thereof. In one embodiment, the nucleic acid sample does not contain a substantial amount, e.g., more than 0.5%, 1%, 2%, 3%, 4%, or 5% of nucleic acids from any other size range or source.

According to yet another aspect of the present invention, it provides an isolated nucleic acid sample useful for genetic testing of a fetus. The nucleic acid sample, e.g., a DNA sample, can be obtained by isolating nucleic acid fragments of from about 50 base pairs to about 100, 200, 300, 400, 500, or 1000 base pairs in length from a cervical mucus sample obtained from a female subject containing the fetus. In one embodiment, these nucleic acid fragments are obtained from the total nucleic acid isolated from the cervical mucus sample by a size fractionation method. In another embodiment, the isolated nucleic acid is substantially free of non-nucleic acid components.

According to still another aspect of the present invention, it provides kits useful for genetic testing or screening of a fetus. In one embodiment, the kit provided by the present invention contains one or more pairs of primers useful for genetic composition testing assays and optionally one or more probes useful for detecting the amplified product(s) by the primers. In another embodiment, the kit provided by the present invention contains one or more pairs of primers useful for testing one or more polymorphisms or genetic markers of a fetus. In yet another embodiment, the kit provided by the present invention contains one or more pairs of primers which are useful for generating amplicons less than about 200 base pairs, less than about 150 base pairs, or between about 75 to about 150 base pairs. In still another embodiment, the kit provided by the present invention contains one or more pairs of primers for one or more designated chromosomes and the primers are selected from the primer sets listed in Tables 2, 3, 4 or 5. In a further embodiment, the kit of the invention contains the pairs of primers of SEQ ID NOs: 1 and 2; SEQ ID NOs: 3 and 4; SEQ ID NOs: 5 and 6; SEQ ID NOs: 9 and 10; SEQ ID NOs: 11 and 12; SEQ ID NOs: 13 and 14; or a combination thereof. The kit can also optionally contain one or more probes and/or other suitable reagents useful for detecting the amplified product(s) by the primers. Further, the kit can comprise instructions for using the pair of primers to test the genetic composition of a fetus.

In still another embodiment, the kit of the present invention contains one or more nucleic acid samples of the present invention. In a further embodiment, the kit provided by the present invention contains the cervical mucus sample of the present invention and an instruction for isolating the nucleic acid sample of the present invention from the cervical mucus sample.

### EXAMPLES

The following examples are intended to illustrate but not to limit the invention in any manner, shape, or form, either explicitly or implicitly. While they are typical of those that might be used, other procedures, methodologies, or techniques known to those skilled in the art may alternatively be used.

### Example 1

This Example describes the collection and isolation of fetal DNA from pregnant women.

Cervical mucous samples were collected from patients, after due consent, by cytobrush method. In the cytobrush method, a Pap smear cytobrush (e.g., MedScand-AB, Malmo, Sweden) was inserted to a maximum depth of 2 cm and removed while rotating it a full turn (i.e., 360°). In order to remove the transcervical cells caught on the brush, the brush was shaken into a test tube containing 2-3 ml of a tissue culture medium (e.g., RPMI-1640 medium, available ATCC, Virginia) in the presence of 1% Penicillin Streptomycin antibiotic. In order to concentrate the transcervical cells on microscopic slides cytospin slides were prepared using e.g., a Cytofunnel Chamber Cytocentrifuge (Thermo-Shandon, England). The conditions used for cytocentrifugation are dependent on the murkiness of the transcervical specimen; if the specimen contained only a few cells, the cells are first centrifuged for five minutes and then suspended with 1 ml of fresh medium. Once prepared, the cytospin slides can be kept in 95% alcohol until further use.

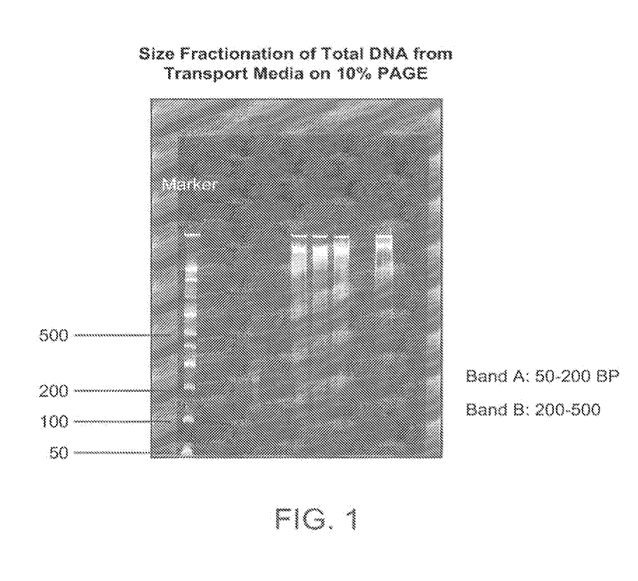

DNA was extracted from fetal tissues, mucous samples or the transport media using Roche's Apoptotic DNA-Ladder Kit following manufacturer's protocol with slight modification. Mucous samples were incubated with equal volume of lysis buffer for 30 minutes to 2 hours or until all the mucous had been dissolved. Some samples needed to be homogenized with a 21 gauze 1.5 inch long needle to facilitate complete mucous dissolution. Total mucous DNA was then size fractionated on 10% PAGE, also known as 10% TBE gel (Invitrogen) under non-denaturing conditions, and the small, 100-250 base pair long DNA band (see FIG. 1) was sliced out after staining the gel with SYBR Gold stain. Fetal DNA from the gel was extracted by soaking the crushed gel in 0.3M sodium acetate (pH 5.5) at 37° C. for overnight followed by desalting the DNA using Promega's Wizard SV Genomic DNA Purification kit.

### Example 2

This Example demonstrates that the DNA obtained from the cervical mucous samples after PAGE purification is indeed fetal DNA.

The total DNA obtained from the cervical swap was size fractionated on 10% PAGE, and the small, 50-250 base pair DNA band (see FIG. 1) was sliced out. The DNA was extracted from PAGE using Promega's Membrane Binding buffer, and its concentration was determined by NanoDrop-1000 Spectrophotometer.

10-20 ng of this size-fractionated DNA was amplified by PCR with primers designed to amplify short STR regions (e.g., D22S1045, CSF1P0, D2S441 see Table 1 for detail).

Typical PCR reaction components were:

| | |
|---|---|
| 10 mM dNTP | 2.0 μl |
| 25 mM MgCl2 | 1.5 μl |
| 50 mM Primers | 0.5 μl |
| Template 1 μg/μl | 2.0 μl |
| Ampli Taq Gold | 0.5 μl |
| 10X PCR Buffer | 2.5 μl |
| Water | 16.0 μl |

Typical PCR cycle consisted of Denaturation temperature of 94° C. for 30 sec, annealing temperature varied from 56 to 62° C. depending upon the primer length, extension was done at 72° C. Number of cycles used ranged from 26 to 40.

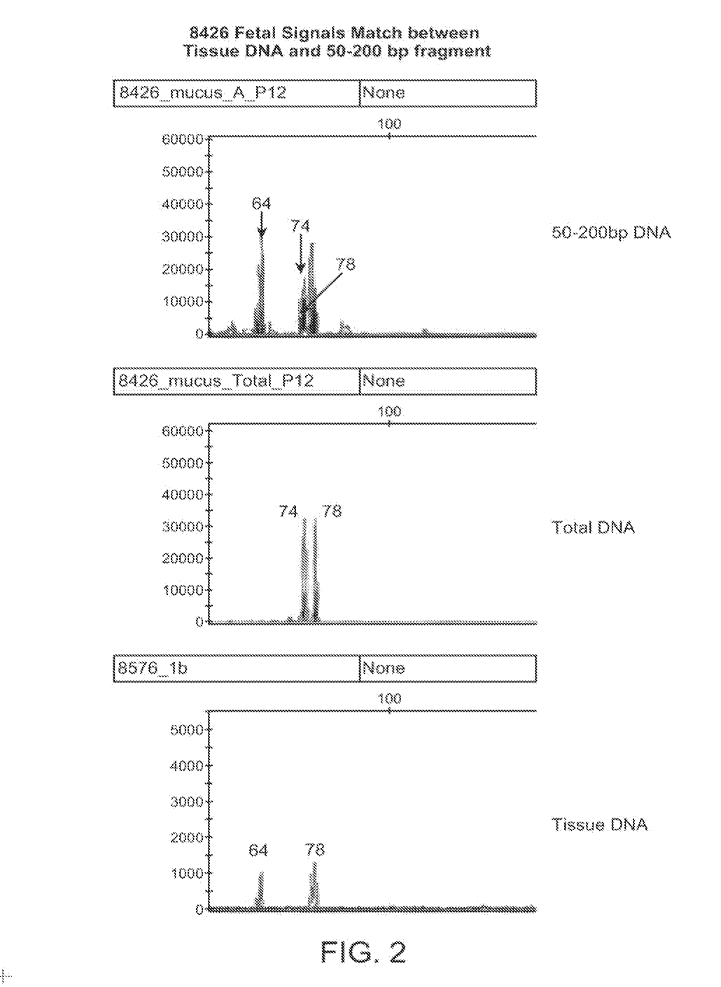

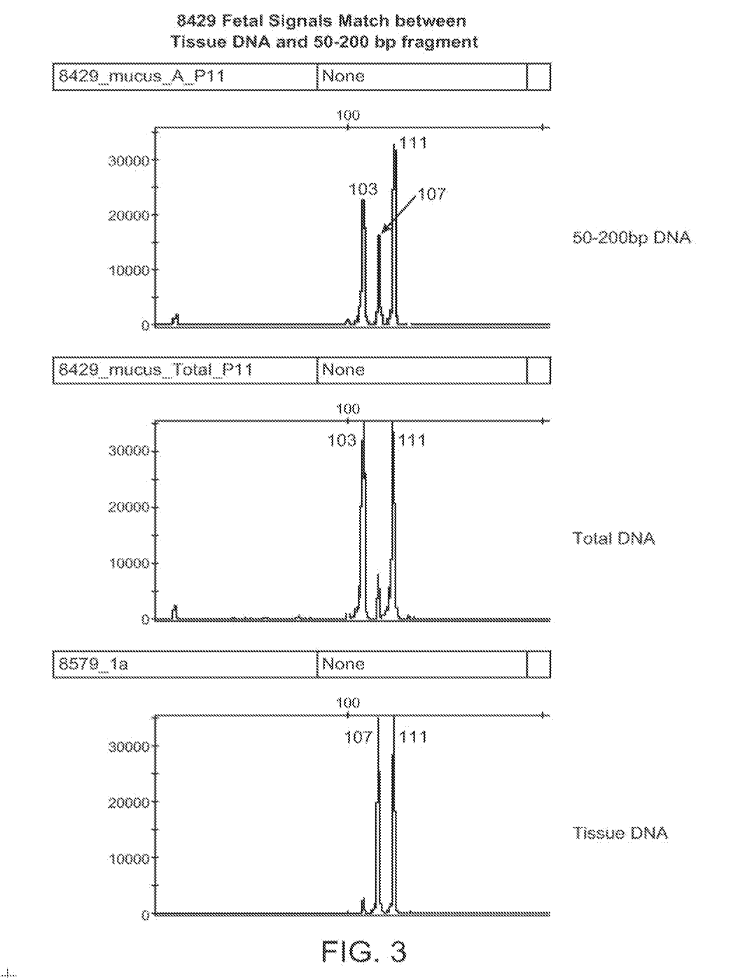

These primers were also used for PCR reaction with the DNA extracted from fetal tissues and the total, unfractionated, mucous DNA. Shown in FIG. 2 and FIG. 3 are PCR electropherograms that demonstrating that the 50-200 base pair DNA fraction resulted in the same fetal alleles as seen in fetal tissue PCR.

### Example 3

This Example demonstrates that the mini-STR markers detect fetal alleles.

Mini-STR markers of the invention were used to detect fetal alleles from DNA extracted from clinical cervical mucous samples. Table 1, below, summarizes the results obtained. D1S1677-F and -R, D22S1045-F and -R, D10S1248-F and -R, TPOX, Mini-LFG33-F and -R, and Mini-LFG34-F and -R are exemplary primers of the invention.

TABLE 1

Detection of Fetal Allele from Clinical Cervical Mucus Samples

| | | |
|---|---|---|
| Sam- | PCR Analysis | Presence |

| ple | Sample | Gel | Informative | | | Maternal | Fetal | of Fetal |
|---|---|---|---|---|---|---|---|---|
| ID | Type | Enriched | Primer Set | Sequence | Seq. ID No. | Alleles | Allele | Allele |
| 7601 | Transport | 8% PAGE | D1S1677-F | FAM-TTCTGTTGGTATAGAGCAGTGTTT | SEQ ID NO: 1 | 90.21, | 99.91 | Yes |
| | Media | | D1S1677-R | GTGACAGGAAGGACGAATG | SEQ ID NO: 2 | 94.91 | | |
| 7602 | Transport | | D22S1045-F | FAM-ATTTTCCCCGATGATAGTAGTCT | SEQ ID NO: 3 | 95.11, | 85.61 | Yes |
| | Media | | D22S1045-R | GCGAATGTATGATTGGCAATATTTTT | SEQ ID NO: 4 | 97.81 | | |
| 7604 | Transport | | D22S1045-F | FAM-ATTTTCCCCGATGATAGTAGTCT | SEQ ID NO: 3 | 98.15 | 92.44 | Yes |
| | Media | | D22S1045-R | GCGAATGTATGATTGGCAATATTTTT | SEQ ID NO: 4 | 102.31 | | |
| | | | D10S1248-F | FAM-TTAATGAATTGAACAAATGAGTGAG | SEQ ID NO: 5 | 104.21 | 10.6, | |
| | | | D10S1248-R | GCAACTCTGGTTGTATTGTCTTCAT | SEQ ID NO: 6 | 111.12 | 55 | |
| 7843 | Transport | | D10S1240-F | TAMRA-TCACCTGTCCCAGCTATCTG | SEQ ID NO: 7 | 101.33 | 104.66 | Yes |
| | Media | | D10S1240-R | AAAATTACTGGTACACTTGATAGCT | SEQ ID NO: 8 | 107.54 | | |
| 7845 | Transport | | D1S1677-F | FAM-TTCTGTTGGTATAGAGCAGTGTTT | SEQ ID NO: 1 | 97.11 | 105.71 | Yes |
| | Media | | D1S1677-R | GTGACAGGAAGGACGAATG | SEQ ID NO: 2 | 101.91 | | |
| 7846 | Transport | | D1S1677-F | FAM-TTCTGTTGGTATAGAGCAGTGTTT | SEQ ID NO: 1 | 93.67 | 46.22 | Yes |
| | Media | | D1S1677-R | GTGACAGGAAGGACGAATG | SEQ ID NO: 2 | 102.33 | | |
| 8034 | Media | | TPOX-F | NED-CTTAGGGAACCCTCACTGAATG | SEQ ID NO: 9 | 148.55, | 160.32 | Yes |
| | | | TPOX-R | GTCCTTGTCAGCGTTTATTTGC | SEQ ID NO: 10 | 156.34 | | |
| 8379 | Mucus | 10% | Profiler-F | commercially available from ABI | | 172.1, | 125, | Yes |
| | | PAGE | Profiler-R | commercially available from ABI | | 176 | 245 | |
| 9558 | Mucous | 1.5% | Mini-LFG34-F | FAM-CACAAGGCAGAATAAAGGGA | SEQ ID NO: 11 | 134, | 142 | Yes |
| | | Agarose | Mini-LFG34-R | TTCATAGTCTGTCTTGTCTTGTCTCA | SEQ ID NO: 12 | 150 | | |
| 9560 | Mucous | 1.5% | Mini-LFG33-F | TAMRA-CACCATACCCAGCCTTACTG | SEQ ID NO: 13 | 118, | 116 | Yes |
| | | Agarose | Mini-LFG33-R | CATGTTACTGTGCTGAATATTGTAGGC | SEQ ID NO: 14 | 122 | | |
| 9561 | Mucous | 1.5% | Mini-LFG34-F | FAM-CACAAGGCAGAATAAAGGGA | SEQ ID NO: 11 | 135, | 146 | Yes |
| | | Agarose | Mini-LFG34-R | TTCATAGTCTGTCTTGTCTTGTCTCA | SEQ ID NO: 12 | 150 | | |

TABLE 2

Mini-STR Primers for Chromosome 21

| | | | | | | |
|---|---|---|---|---|---|---|
| Number | Name/Loci | 5′ Label | Sequence | Seq. ID No. | Length | Chromosome |

| | | | | | | |
|---|---|---|---|---|---|---|
| p21M-1 | Mini LFG20-F | 6-FAM | TTGAGAAGGCCCCACCTATG | SEQ ID NO: 15 | 20 | 21 |
| p21M-2 | Mini LFG20-R | | AGAAGGCCCCCTGTGTTATG | SEQ ID NO: 16 | 20 | 21 |
| p21M-3 | Mini LFG21-F | HEX | GCGAATCATGACACTAATTTTGG | SEQ ID NO: 17 | 23 | 21 |
| p21M-4 | Mini LFG21-R | | TGGAGAAGAAAAAGAGGCCTGA | SEQ ID NO: 18 | 22 | 21 |
| p21M-5 | Mini LFG24-F | NED | GGTTCTTTGGAAAATTGTTTAGGC | SEQ ID NO: 19 | 24 | 21 |
| p21M-6 | Mini LFG24-R | | TGCTCTGGACTTACAGCATCAA | SEQ ID NO: 20 | 22 | 21 |
| p21M-7 | Mini LFG26-F | 6-FAM | CCCTTAAAACCATATTTTTCACCTC | SEQ ID NO: 21 | 25 | 21 |
| p21M-8 | Mini LFG26-R | | AGCCTGGGTGACAGAGCAAG | SEQ ID NO: 22 | 20 | 21 |
| p21M-9 | Mini LFG29-F | HEX | TTGCTTGAGAGGTAAAAAGAAAA | SEQ ID NO: 23 | 23 | 21 |
| p21M-10 | Mini LFG29-R | | GAGCAACAGAGCGAGATTCTG | SEQ ID NO: 24 | 21 | 21 |
| p21M-11 | Mini LFG33-F | NED | CACCATACCCAGCCTTACTG | SEQ ID NO: 25 | 20 | 21 |
| p21M-12 | Mini LFG33-R | | CATGTTACTGTGCTGAATATTGTAGGC | SEQ ID NO: 26 | 27 | 21 |
| p21M-13 | Mini LFG34-F | 6-FAM | GGCAGAATAAAGGGATTATTGC | SEQ ID NO: 27 | 22 | 21 |
| p21M-14 | Mini LFG34-R | | TTCATAGTCTGTCTTGTCTTGTCTCA | SEQ ID NO: 28 | 26 | 21 |
| p21M-15 | Mini D21S2054-F | NED | GCAGTAAATGTCTATGAAACAAGG | SEQ ID NO: 29 | 24 | 21 |
| p21M-16 | Mini D21S2054-R | | TGATAAATAGTGAATATAGTTGACAGC | SEQ ID NO: 30 | 27 | 21 |
| p21M-17 | Mini D21S1904-F | * | CAACAATTCCTTCTAATTTTCCA | SEQ ID NO: 31 | 23 | 21 |
| p21M-18 | Mini D21S1904-R | | TGTCTGGTTTCCCCATCTCT | SEQ ID NO: 32 | 20 | 21 |
| p21M-19 | Mini D21S1911-F | * | TGAGGAGACATCCTTGACAAAA | SEQ ID NO: 33 | 22 | 21 |
| p21M-20 | Mini D21S1911-R | | CATACACACAGCAAGTATGAGTGA | SEQ ID NO: 34 | 24 | 21 |
| p21M-21 | Mini D21S1256-F | * | GCCTATGGTCCCATCATAACA | SEQ ID NO: 35 | 21 | 21 |
| p21M-22 | Mini D21S1256-R | | TCCACAGTTCTTAGATGGCTTT | SEQ ID NO: 36 | 22 | 21 |
| p21M-23 | Mini D21S1899-F | * | TGAAAACGTGTTGACAGATGAA | SEQ ID NO: 37 | 22 | 21 |
| p21M-24 | Mini D21S1899-R | | AATGGCAGGATTTTCTTTTT | SEQ ID NO: 38 | 20 | 21 |
| p21M-25 | Mini D21S1922-F | * | TGTCAAAATATGTGGATTAGACAAAA | SEQ ID NO: 39 | 26 | 21 |
| p21M-26 | Mini D21S1922-R | | TCACTGGTATACTGTGATGTGTGC | SEQ ID NO: 40 | 24 | 21 |
| p21M-27 | Mini D21S1884-F | * | AAAAATTATTGATAACGTTCAGTAT | SEQ ID NO: 41 | 25 | 21 |
| p21M-28 | Mini D21S1884-R | | TTTCTAACAATATGTACCCACTGGA | SEQ ID NO: 42 | 25 | 21 |
| p21M-29 | Mini D21S1914-F | * | CATTGGGCCTTCTGTCAAAT | SEQ ID NO: 43 | 20 | 21 |
| p21M-30 | Mini D21S1914 R | | TCTGCAGAATTTCATTTGCTGT | SEQ ID NO: 44 | 22 | 21 |
| p21M-31 | Mini D21S263-F | * | CAAACTTGAAATATGAAAAAGTCATCA | SEQ ID NO: 45 | 27 | 21 |
| p21M-32 | Mini D21S263-R | | TTTCTGTATTTCCTGAAACAACATTT | SEQ ID NO: 46 | 26 | 21 |
| p21M-33 | Mini D21S1252-F | * | TCTGTCTTTGTCTCACTATCTGTCTG | SEQ ID NO: 47 | 26 | 21 |
| p21M-34 | Mini D21S1252-R | | TAGGGTGAGGACCCCTTTCT | SEQ ID NO: 48 | 20 | 21 |
| p21M-35 | Mini D21S1919-F | * | CCTGGATTATTTGTTCAAAGTCAG | SEQ ID NO: 49 | 24 | 21 |
| p21M-36 | Mini D21S1919-R | | TCTCATGTTCCTTGGCCTGT | SEQ ID NO: 50 | 20 | 21 |
| p21M-37 | Mini D21S1255-F | * | ATTTTGCCACATAGAGAAAAATA | SEQ ID NO: 51 | 23 | 21 |
| p21M-38 | Mini D21S1255-R | | GCCTGGACATCCTCTTTCT | SEQ ID NO: 52 | 19 | 21 |
| p21M-39 | Mini D21S266-F | * | AGATGTAGCACAGTTAGATGCAGA | SEQ ID NO: 53 | 24 | 21 |
| p21M-40 | Mini D21S266-R | | AGCAGAAAAAGCCATTCTGG | SEQ ID NO: 54 | 20 | 21 |
| p21M-41 | Mini D21S2058-F | * | GTCATGACCCTGGCTGTG | SEQ ID NO: 55 | 18 | 21 |
| p21M-42 | Mini D21S2058-R | | AGGGCAGGCTGTGCTCAT | SEQ ID NO: 56 | 18 | 21 |
| p21M-43 | Mini D21S1431-F | * | GGGACCATTTTAGATATTCTGCT | SEQ ID NO: 57 | 23 | 21 |
| p21M-44 | Mini D21S1431-R | | GCACTTAACAAGCACTGAATCAA | SEQ ID NO: 58 | 23 | 21 |
| p21M-45 | Mini D21S259-F | * | TCCTGAAGGAAGAATGTGGTC | SEQ ID NO: 59 | 21 | 21 |
| p21M-46 | Mini D21S259-R | | ATGCATGGTCGTGTGTGTG | SEQ ID NO: 60 | 19 | 21 |
| p21M-47 | Mini D21S270-F | * | TTTTTCAAAATCAAAAAGATAGTGA | SEQ ID NO: 61 | 25 | 21 |
| p21M-48 | Mini D21S270-R | | GGAGGGCATCTGGGTAATTT | SEQ ID NO: 62 | 20 | 21 |
| p21M-49 | Mini D21S1912-F | * | GCCATCAGCCCTCATACAGA | SEQ ID NO: 63 | 20 | 21 |
| p21M-50 | Mini D21S1912-R | | GAATTTGGGGGACGCAGT | SEQ ID NO: 64 | 18 | 21 |
| p21M-51 | Mini D21S260-F | * | CGAGAAGTTTCCCATGCATTT | SEQ ID NO: 65 | 21 | 21 |
| p21M-52 | Mini D21S260-R | | AAATTCAGTGATGGGAAGAAGG | SEQ ID NO: 66 | 22 | 21 |
| p21M-53 | Mini D21S261-F | * | CCTAAAACAGCATCAACAGAAA | SEQ ID NO: 67 | 22 | 21 |
| p21M-54 | Mini D21S261-R | | TTGGACCTTTTGATTTTTCCT | SEQ ID NO: 68 | 21 | 21 |
| p21M-55 | Mini D21S262-F | * | CAGCAACTCCCACTTCTGAC | SEQ ID NO: 69 | 20 | 21 |
| p21M-56 | Mini D21S262-R | | TTGTTGTTGAGTGAAAGAATAGAGAAA | SEQ ID NO: 70 | 27 | 21 |
| p21M-57 | Mini D21S1892-F | * | AAATCTGAATTATGTCCAATCAAAA | SEQ ID NO: 71 | 25 | 21 |
| p21M-58 | Mini D21S1892-R | | TGAGTTTTTGGAAGAGAGAGAGAGA | SEQ ID NO: 72 | 25 | 21 |
| p21M-59 | Mini D21S272-F | * | AAAAGGGGATCCAATATGAAA | SEQ ID NO: 73 | 21 | 21 |
| p21M-60 | Mini D21S272-R | | TGGAACAATTTTATCCTTAGTTTGTC | SEQ ID NO: 74 | 26 | 21 |
| p21M-61 | Mini D21S1893-F | * | TATGCACACCACACGGACAC | SEQ ID NO: 75 | 20 | 21 |
| p21M-62 | Mini D21S1893-R | | GTTCCGGGGAAGTTTTATGC | SEQ ID NO: 76 | 20 | 21 |
| p21M-63 | Mini D21S265-F | * | TGGCAAAGAGAACAACAGCA | SEQ ID NO: 77 | 20 | 21 |
| p21M-64 | Mini D21S265-R | | TTCTGTGAATATGGGTTCTGGA | SEQ ID NO: 78 | 22 | 21 |
| p21M-65 | Mini D21S267-F | * | GGGGATTATTTATGTAGAAAATGAGA | SEQ ID NO: 79 | 26 | 21 |
| p21M-66 | Mini D21S267-R | | GGTGACAGACCCTGTCTCTAAAA | SEQ ID NO: 80 | 23 | 21 |
| p21M-67 | Mini D21S268-F | * | TGGGCAACAGAGTGAGACAG | SEQ ID NO: 81 | 20 | 21 |
| p21M-68 | Mini D21S268-R | | CACATCCTTGCCAACACTTG | SEQ ID NO: 82 | 20 | 21 |
| p21M-69 | Mini D21S269-F | * | GATACTGAATCATCCCTTTCATTC | SEQ ID NO: 83 | 24 | 21 |
| p21M-70 | Mini D21S269-R | | TTCCGTTATTAATTTTATTCTGAGG | SEQ ID NO: 84 | 25 | 21 |
| p21M-71 | Mini D21S1902-F | * | GAGTGAGAGACAGACAGAGAGACG | SEQ ID NO: 85 | 24 | 21 |
| p21M-72 | Mini D21S1902-R | | CAGTAGGGGCAGACTATTTTACTC | SEQ ID NO: 86 | 24 | 21 |
| p21M-73 | Mini D21S1253-F | * | ATGGGTGACAGAGCGAGACT | SEQ ID NO: 87 | 20 | 21 |
| p21M-74 | Mini D21S1253-R | | TTCAGAGCCTGGGTTAAACA | SEQ ID NO: 88 | 20 | 21 |
| p21M-75 | Mini D21S1254-F | * | AATGACCATCCTTAAAACAACTTT | SEQ ID NO: 89 | 24 | 21 |
| p21M-76 | Mini D21S1254-R | | GTGGCTGAGCGAGACTCTGT | SEQ ID NO: 90 | 20 | 21 |
| p21M-77 | Mini D21S1907-F | * | TCACTCAATTTATGAGAGCGAAA | SEQ ID NO: 91 | 23 | 21 |
| p21M-78 | Mini D21S1907-R | | TCAGCCTTTGATATGTGCATT | SEQ ID NO: 92 | 21 | 21 |
| p21M-79 | Mini D21S1909-F | * | GCATGAGTGGAAAAATGTGAAA | SEQ ID NO: 93 | 22 | 21 |
| p21M-80 | Mini D21S1909-R | | CTGAGTCAAGAGCAGGCAACT | SEQ ID NO: 94 | 21 | 21 |
| p21M-81 | Mini D21S1910-F | * | CCAATGCTTTTGATTTTTAAGC | SEQ ID NO: 95 | 22 | 21 |
| p21M-82 | Mini D21S1910-R | | CGGCAAAGTAGTATTTAATGTGCT | SEQ ID NO: 96 | 24 | 21 |
| p21M-83 | Mini D21S1257-F | * | TTTCATTCACCGGTTTTCCT | SEQ ID NO: 97 | 20 | 21 |
| p21M-84 | Mini D21S1257-R | | TTTTAGGGTATATCTGCCATAATGC | SEQ ID NO: 98 | 25 | 21 |
| p21M-85 | Mini D21S1258-F | * | GGGAAGGAAAATAAAGCATTGA | SEQ ID NO: 99 | 22 | 21 |
| p21M-86 | Mini D21S1258-R | | GCACAAAAACAAAATCTGTCACT | SEQ ID NO: 100 | 23 | 21 |
| p21M-87 | Mini D21S1913-F | * | TTGCTGGGTTGTTAAACTTATTCA | SEQ ID NO: 101 | 24 | 21 |
| p21M-88 | Mini D21S1913-R | | AAAGGTGAACGCTGGTATCG | SEQ ID NO: 102 | 20 | 21 |
| p21M-89 | Mini D21S1259-F | * | GACCCCAACACTGGACACA | SEQ ID NO: 103 | 19 | 21 |
| p21M-90 | Mini D21S1259-R | | CTTGGTAAGTGGGCAGTGAG | SEQ ID NO: 104 | 20 | 21 |
| p21M-91 | Mini D21S1915-F | * | CATGCTCATAGATATGCACACA | SEQ ID NO: 105 | 22 | 21 |
| p21M-92 | Mini D21S1915-R | | GATTGTGCCAGGTCTCAGGT | SEQ ID NO: 106 | 20 | 21 |
| p21M-93 | Mini D21S1916-F | * | CCAAGGTGAATTCCCAATTT | SEQ ID NO: 107 | 20 | 21 |
| p21M-94 | Mini D21S1916-R | | GCGGCACATTTTCACAGACT | SEQ ID NO: 108 | 20 | 21 |
| p21M-95 | Mini D21S1917-F | * | TGAACATTAACACTGGTAACTTTACAT | SEQ ID NO: 109 | 27 | 21 |
| p21M-96 | Mini D21S1917-R | | TGTCCTCTCCATTTTGCTTG | SEQ ID NO: 110 | 20 | 21 |
| p21M-97 | Mini D21S1918-F | * | CTTCTCTCCAATTCCCATGC | SEQ ID NO: 111 | 20 | 21 |
| p21M-98 | Mini D21S1918-R | | GAAGGAAAAATTGGGATTTCG | SEQ ID NO: 112 | 21 | 21 |
| p21M-99 | Mini D21S1920-F | * | CAGCCTGGGTGACAGAGAC | SEQ ID NO: 113 | 19 | 21 |
| p21M-100 | Mini D21S1920-R | | TGTTGATGAAGCATTTACTCATACAT | SEQ ID NO: 114 | 26 | 21 |
| p21M-101 | Mini D21S1921-F | * | TCCCCCTAAATGGACAACTTT | SEQ ID NO: 115 | 21 | 21 |
| p21M-102 | Mini D21S1921-R | | GCTTTGTTTTCCTTTAGCTTCC | SEQ ID NO: 116 | 22 | 21 |
| p21M-103 | Mini D21S1883-F | * | TCTGGAATGGTTAAGGCAGAA | SEQ ID NO: 117 | 21 | 21 |
| p21M-104 | Mini D21S1883-R | | CACCATTCTCCCTAGCATGA | SEQ ID NO: 118 | 20 | 21 |
| p21M-105 | Mini D21S1885-F | * | CAAGGTGGAAGGCAGAAGG | SEQ ID NO: 119 | 19 | 21 |
| p21M-106 | Mini D21S1885-R | | CGCGCTCTCTCTCTCTCTCT | SEQ ID NO: 120 | 20 | 21 |
| p21M-107 | Mini D21S1886-F | * | TGCAAGATTTCCCCCTTCTA | SEQ ID NO: 121 | 20 | 21 |
| p21M-108 | Mini D21S1886-R | | CTTTGACTCCTCAGCCGTGT | SEQ ID NO: 122 | 20 | 21 |
| p21M-109 | Mini D21S1887-F | * | CCTGGCATCTCTGTTTTTA | SEQ ID NO: 123 | 19 | 21 |
| p21M-110 | Mini D21S1887-R | | AAAGATGATGTCAGGAATGC | SEQ ID NO: 124 | 20 | 21 |
| p21M-111 | Mini D21S1260-F | * | CATCCAAGGGGAACACAAGT | SEQ ID NO: 125 | 20 | 21 |
| p21M-112 | Mini D21S1260-R | | GGAAGTAAAAGGCCCAGAGG | SEQ ID NO: 126 | 20 | 21 |
| p21M-113 | Mini D21S1888-F | * | AAAAAGGATGTTTGTTATGGTAAGA | SEQ ID NO: 127 | 25 | 21 |
| p21M-114 | Mini D21S1888-R | | GAACTGTGTGCCAGGAACTG | SEQ ID NO: 128 | 20 | 21 |
| p21M-115 | Mini D21S1889-F | * | TGTGTGTTTGCATGTATGTGTAGA | SEQ ID NO: 129 | 24 | 21 |
| p21M-116 | Mini D21S1889-R | | ACAGAAACATGGCTGCCTCT | SEQ ID NO: 130 | 20 | 21 |
| p21M-117 | Mini D21S1890-F | * | GACCACAGATTTCCCAATCG | SEQ ID NO: 131 | 20 | 21 |
| p21M-118 | Mini D21S1890-R | | AAACCAACTGACTCCCAAACA | SEQ ID NO: 132 | 21 | 21 |
| p21M-119 | Mini D21S1891-F | * | CAGAGCGAGACTCCATCTCA | SEQ ID NO: 133 | 20 | 21 |
| p21M-120 | Mini D21S1891-R | | GGAACCCTTGGATGTTGCTA | SEQ ID NO: 134 | 20 | 21 |
| p21M-121 | Mini D21S1894-F | * | GCTCAATGCTATTGGAGTGC | SEQ ID NO: 135 | 20 | 21 |
| p21M-122 | Mini D21S1894-R | | TTCACAAAACAGAACCAACCA | SEQ ID NO: 136 | 21 | 21 |
| p21M-123 | Mini D21S1895-F | * | TCTCCCTTGCCAGACTTCTC | SEQ ID NO: 137 | 20 | 21 |
| p21M-124 | Mini D21S1895-R | | GCCCAGGATGGACAAAGA | SEQ ID NO: 138 | 18 | 21 |
| p21M-125 | Mini D21S1896-F | * | CACATGTGGCCACTGCAC | SEQ ID NO: 139 | 18 | 21 |
| p21M-126 | Mini D21S1896-R | | CCAGGGATTTGTCTAGAAAGGA | SEQ ID NO: 140 | 22 | 21 |
| p21M-127 | Mini D21S1897-F | * | GCCTAGGCAACCAGAGTGAG | SEQ ID NO: 141 | 20 | 21 |
| p21M-128 | Mini D21S1897-R | | TGTATTTCTTTTCTTTTTAAATGGTGA | SEQ ID NO: 142 | 27 | 21 |
| p21M-129 | Mini D21S1898-F | * | CTCTTCAGCAGCCGAGAAAA | SEQ ID NO: 143 | 20 | 21 |
| p21M-130 | Mini D21S1898-R | | CACCAGAAAGCAAGGAAGGA | SEQ ID NO: 144 | 20 | 21 |
| p21M-131 | Mini D21S1900-F | * | ACACCAGAGGGCAGAGTGAG | SEQ ID NO: 145 | 20 | 21 |
| p21M-132 | Mini 021S1900-R | | AAAGCACTGTATGTTTCTGTGAGA | SEQ ID NO: 146 | 24 | 21 |
| p21M-133 | Mini D21S1901-F | * | GGTTGTCCAGAGAAACAACCA | SEQ ID NO: 147 | 21 | 21 |
| p21M-134 | Mini D21S1901-R | | CAATTGTGTGAGCCAATCTCTC | SEQ ID NO: 148 | 22 | 21 |
| p21M-135 | Mini D21S1903-F | * | GGCAATTCCTTAAAATAAATCACTC | SEQ ID NO: 149 | 25 | 21 |
| p21M-136 | Mini D21S1903-R | | GCCCGGCCCTATCTATCTAT | SEQ ID NO: 150 | 20 | 21 |
| p21M-137 | Mini D21S1905-F | * | GCATGCTCGCTCTCTCTCTT | SEQ ID NO: 151 | 20 | 21 |
| p21M-138 | Mini D21S1905-R | | AACTGGCGTGTCTACACCATC | SEQ ID NO: 152 | 21 | 21 |
| p21M-139 | Mini D21S1906-F | * | GAGCAAGACTCCATCTCAAAAA | SEQ ID NO: 153 | 22 | 21 |
| p21M-140 | Mini D21S1906-R | | AAAGATTGCCCAACAAATGG | SEQ ID NO: 154 | 20 | 21 |
| p21M-141 | Mini D21S1908-F | * | TGGGTTCCATAGTTCTAATGTGTG | SEQ ID NO: 155 | 24 | 21 |
| p21M-142 | Mini D21S1908-R | | TCTCTGGCTGGACATACTATTCA | SEQ ID NO: 156 | 23 | 21 |
| p21M-143 | Mini D21S1438-F | * | GGAGAAGGTAGCTAAAGGATGAAA | SEQ ID NO: 157 | 24 | 21 |
| p21M-144 | Mini D21S1438-R | | TGCACCCTAGGACCTAAAGAA | SEQ ID NO: 158 | 21 | 21 |
| p21M-145 | Mini D21S1439-F | * | CCTGATCTGGTCCTGTAGCC | SEQ ID NO: 159 | 20 | 21 |
| p21M-146 | Mini D21S1439-R | | TGAGTTTGAAAATAAAGTGTTCTGC | SEQ ID NO: 160 | 25 | 21 |
| p21M-147 | Mini ATA42C09-F | * | CAAAGCGAGACCTTTTCTCAA | SEQ ID NO: 161 | 21 | 21 |
| p21M-148 | Mini ATA42C09-R | | CGAGTCATAGATCCATTACCCATT | SEQ ID NO: 162 | 24 | 21 |
| p21M-149 | Mini D21S1441-F | * | ACAACCGAGCGAGACCTG | SEQ ID NO: 163 | 18 | 21 |
| p21M-150 | Mini D21S1441-R | | GGAACTGATGGTCACAAGATAGTC | SEQ ID NO: 164 | 24 | 21 |
| p21M-151 | Mini D21S120-F | * | TTGTGGATTTTCCCAATTGAT | SEQ ID NO: 165 | 21 | 21 |
| p21M-152 | Mini D21S120-R | | TTTGTATTTGCCTAAAAACAGAGC | SEQ ID NO: 166 | 24 | 21 |
| p21M-153 | Mini D21S167-F | * | TACCAGCTTCAAACGTGCAG | SEQ ID NO: 167 | 20 | 21 |
| p21M-154 | Mini D21S167-R | | CCTTGCCCTGAAGCACAT | SEQ ID NO: 168 | 18 | 21 |
| p21M-155 | Mini D21S168-F | * | TGTAGGCTGTTTAGTTGGTGGA | SEQ ID NO: 169 | 22 | 21 |
| p21M-156 | Mini D21S168-R | | CGGCATCACAGTCTGATAAAA | SEQ ID NO: 170 | 21 | 21 |
| p21M-157 | Mini D21S210-F | * | TGATAAGCCTCCCTCACTACTATTTT | SEQ ID NO: 171 | 26 | 21 |
| p21M-158 | Mini D21S210-R | | GCAGCGATAGCTAGTCATAGTGAA | SEQ ID NO: 172 | 24 | 21 |
| p21M-159 | Mini D21S214-F | * | CCTGCAAGGACACCAAGTTA | SEQ ID NO: 173 | 20 | 21 |
| p21M-160 | Mini D21S214-R | | TGTTCACCTGATTTTCGGTTC | SEQ ID NO: 174 | 21 | 21 |
| p21M-161 | Mini GATA116E08-F | * | TGCAAGCCACATCATTTGT | SEQ ID NO: 175 | 19 | 21 |
| p21M-162 | Mini GATA116E08-R | | TCGAATCGATAGATAGATAGGTGA | SEQ ID NO: 176 | 24 | 21 |
| p21M-163 | Mini GATA148F04-F | * | TGAAACAAGGGAATCTATCATC | SEQ ID NO: 177 | 22 | 21 |
| p21M-164 | Mini GATA148F04-R | | TGATAAATAGTGAATATAGTTGACAGC | SEQ ID NO: 178 | 27 | 21 |
| p21M-165 | Mini GATA163G03-F | * | TTGTGGGGCCTTGTAATTGT | SEQ ID NO: 179 | 20 | 21 |
| p21M-166 | Mini GATA163G03-R | | CAGGGTCCCTAGAGAGACAGA | SEQ ID NO: 180 | 21 | 21 |
| p21M-167 | Mini D21S2055-F | * | CAGAACCAATAGGCTATCTATCT | SEQ ID NO: 181 | 23 | 21 |
| p21M-168 | Mini D21S2055-R | | ACAGTAAATCACTTGGTAGGAGA | SEQ ID NO: 182 | 23 | 21 |
| p21M-169 | Mini D21S1442-F | * | GGGCACCCCTTTATACTTGG | SEQ ID NO: 183 | 20 | 21 |
| p21M-170 | Mini D21S1442-R | | TCACATGAGCCAATTCCTTATAATAG | SEQ ID NO: 184 | 26 | 21 |
| p21M-171 | Mini GATA29C02-F | * | TCTATACATATGTGTGTGTGCAT | SEQ ID NO: 185 | 23 | 21 |
| p21M-172 | Mini GATA29C02-R | | CACCTTTGTTGCCAAGAGTC | SEQ ID NO: 186 | 20 | 21 |
| p21M-173 | Mini GATA29D01-F | * | TTCTGTTAAATGAGTAAGGAGATGACA | SEQ ID NO: 187 | 27 | 21 |
| p21M-174 | Mini GATA29D01-R | | GCATGCGTGTGTGTGTGTAT | SEQ ID NO: 188 | 20 | 21 |
| p21M-175 | Mini D21S1433-F | * | GAGCTGAGATCACGACAGTCA | SEQ ID NO: 189 | 21 | 21 |
| p21M-176 | Mini D21S1433-R | | TATTTTCAGGCCAAGCCTTT | SEQ ID NO: 190 | 20 | 21 |
| p21M-177 | Mini GATA45C03-F | * | GAAACAGAACTAATAGGATCTATCTGC | SEQ ID NO: 191 | 27 | 21 |
| p21M-178 | Mini GATA45C03-R | | GGCAAACAAATAGTTGATAGATGAG | SEQ ID NO: 192 | 25 | 21 |
| p21M-179 | Mini D21S1446-F | * | TGACCATCTTACTGGTTTATGTATTT | SEQ ID NO: 193 | 26 | 21 |
| p21M-180 | Mini D21S1446-R | | CGAGGCTATTTTACTGGTAACTAACTG | SEQ ID NO: 194 | 27 | 21 |
| p21M-181 | Mini D21S1270-F | * | GGGCTACATAGAGAAACAGAACC | SEQ ID NO: 195 | 23 | 21 |
| p21M-182 | Mini D21S1270-R | | ACACACACACACACACATGC | SEQ ID NO: 196 | 20 | 21 |
| p21M-183 | Mini D21S1436-F | * | GGAAAGAGAAAGAAAGGAAGGAA | SEQ ID NO: 197 | 23 | 21 |
| p21M-184 | Mini D21S1436-R | | CCATTTATGTCCTATTTCCTACTCC | SEQ ID NO: 198 | 25 | 21 |
| p21M-185 | Mini D21S1265-F | * | GGACTCCTCCAGCTGAACTCT | SEQ ID NO: 199 | 21 | 21 |
| p21M-186 | Mini D21S1265-R | | GCACAGTACAGCAAACTTGTCA | SEQ ID NO: 200 | 22 | 21 |
| p21M-187 | Mini D21S1249-F | * | TTTCCACACGGCTAATCTACTT | SEQ ID NO: 201 | 22 | 21 |
| p21M-188 | Mini D21S1249-R | | TACCTCCCTCCCTCCATCC | SEQ ID NO: 202 | 19 | 21 |
| p21M-189 | Mini D21S1409-F | * | GGGGAATACATTTGTGTAGGTAGG | SEQ ID NO: 203 | 24 | 21 |
| p21M-190 | Mini D21S1409-R | | CACTAATACCTGGTGAATGATTCTT | SEQ ID NO: 204 | 25 | 21 |
| p21M-191 | Mini D21S1410-F | * | AAATGAAGATATTTTCTTAGCTTAT | SEQ ID NO: 205 | 25 | 21 |
| p21M-192 | Mini D21S1410-R | | GCATTCAATATTTTACTTTTAAGAATC | SEQ ID NO: 206 | 27 | 21 |
| p21M-193 | Mini D21S1250-F | * | GGGTAAAGAAAATGTGCTCTCTC | SEQ ID NO: 207 | 23 | 21 |
| p21M-194 | Mini D21S1250-R | | GGTTCTCCAAGTTCAATGGTG | SEQ ID NO: 208 | 21 | 21 |
| p21M-195 | Mini D21S1251-F | * | CAGCAGAAAGGGAATAGTTGG | SEQ ID NO: 209 | 21 | 21 |
| p21M-196 | Mini D21S1251-R | | CAAGTTAAAAACACAAAATGGAAA | SEQ ID NO: 210 | 24 | 21 |
| p21M-197 | Mini D21S1411-F | * | TGGATGGATGGATAGATACACAG | SEQ ID NO: 211 | 23 | 21 |
| p21M-198 | Mini D21S1411-R | | CCCACTCCCAGCCTTCTAA | SEQ ID NO: 212 | 19 | 21 |
| p21M-199 | Mini D21S1238-F | * | TCTCTGTGTCTATGTGTGCATGTT | SEQ ID NO: 213 | 24 | 21 |
| p21M-200 | Mini D21S1238-R | | GGTTTTGCAAAGGCAGGTTA | SEQ ID NO: 214 | 20 | 21 |
| p21M-201 | Mini D21S1239-F | * | CACTTTCACAATATGTATTGCTTATCA | SEQ ID NO: 215 | 27 | 21 |
| p21M-202 | Mini D21S1239-R | | GGTAGCAATTTCACTCTCTCTTTC | SEQ ID NO: 216 | 24 | 21 |
| p21M-203 | Mini D21S1408-F | * | GATGACAAGACAGATTAGATAGATTGG | SEQ ID NO: 217 | 27 | 21 |
| p21M-204 | Mini D21S1408-R | | CATTGGGCTTATTTTTCTTTCTAT | SEQ ID NO: 218 | 24 | 21 |
| p21M-205 | Mini UT556-F | * | AAAGGCAGGAAGGCAGGA | SEQ ID NO: 219 | 18 | 21 |
| p21M-206 | Mini UT556-R | | TTTTCTTCTTTTTGCTTCTCTTTTC | SEQ ID NO: 220 | 25 | 21 |
| p21M-207 | Mini D21S1240-F | * | TGATGTAGTTCACTAGGATGTAGGG | SEQ ID NO: 221 | 25 | 21 |
| p21M-208 | Mini D21S1240-R | | CCTGAGACACAAGAGCGAGA | SEQ ID NO: 222 | 20 | 21 |
| p21M-209 | Mini D21S1412-F | * | CCTGGGTGACAAGAGTGAAA | SEQ ID NO: 223 | 20 | 21 |
| p21M-210 | Mini D21S1412-R | | CACAGAAATTTGTAGAACCACAGC | SEQ ID NO: 224 | 24 | 21 |
| p21M-211 | Mini D21S1280-F | * | GGCATCAAAATTTGGAAGAAA | SEQ ID NO: 225 | 21 | 21 |
| p21M-212 | Mini D21S1280-R | | AAAGCTGAGCTGAATGGTGA | SEQ ID NO: 226 | 20 | 21 |
| p21M-213 | Mini D21S1413-F | * | GGGAAACCACAGTTATACATTCC | SEQ ID NO: 227 | 23 | 21 |
| p21M-214 | Mini D21S1413-R | | TGTTTACAGTTCTTCACAGAGTTCTT | SEQ ID NO: 228 | 26 | 21 |
| p21M-215 | Mini D21S1244-F | * | ACCACAGAATTCAGTCCAAAAA | SEQ ID NO: 229 | 22 | 21 |
| p21M-216 | Mini D21S1244-R | | TTATCCCCTGGAGGAACTTG | SEQ ID NO: 230 | 20 | 21 |
| p21M-217 | Mini D21S1245-F | * | CCAGAAAATGACACATGAAGGA | SEQ ID NO: 231 | 22 | 21 |
| p21M-218 | Mini D21S1245-R | | GAGATATTGGCCTGTAGTTTTGTTTT | SEQ ID NO: 232 | 26 | 21 |
| p21M-219 | Mini D21S1414-F | * | GATGTTGTATTAGTCAATGTTCTCCA | SEQ ID NO: 233 | 26 | 21 |
| p21M-220 | Mini D21S1414-R | | TCTGTCTGTCTGTCTGTCTGTCTG | SEQ ID NO: 234 | 24 | 21 |
| p21M-221 | Mini D21S1246-F | * | ATGGGCAAACAGATGGGTAG | SEQ ID NO: 235 | 20 | 21 |
| p21M-222 | Mini D21S1246-R | | CCATATTATCATCCATCCATCCA | SEQ ID NO: 236 | 23 | 21 |

Note:.
* = 6-FAM, VIC, NED, JOE, ROX, PET, TAMRA or 5-FAM

TABLE 3

Mini-STR Primers for Chromosome 13

| | | | | | | |
|---|---|---|---|---|---|---|
| Number | Name/Loci | 5′ Label | Sequence | Seq. ID No. | Length | Chromosome |

| | | | | | | |
|---|---|---|---|---|---|---|
| p13M-1 | Mini D13S1236-F | * | GGGTAACAGCATAAGACCCTGT | SEQ ID NO: 237 | 22 | 13 |
| p13M-2 | Mini D13S1236-R | | TCACTTTGGTGCTTGCTTTG | SEQ ID NO: 238 | 20 | 13 |
| p13M-3 | Mini D13S175-F | * | GAATCTGCTGAGAGAGTAGATTTTAAG | SEQ ID NO: 239 | 27 | 13 |
| p13M-4 | Mini D13S175-R | | TGCATCACCTCACATAGGTTACT | SEQ ID NO: 240 | 23 | 13 |
| p13M-5 | Mini D13S1243-F | * | TGCTGACAGGCTACAGAACTTT | SEQ ID NO: 241 | 22 | 13 |
| p13M-6 | Mini D13S1243-R | | TCTGCATTTGTAGAAATAATCTTATCA | SEQ ID NO: 242 | 27 | 13 |
| p13M-7 | Mini D13S1304-F | * | ACCATTATTCTCCTGAGTCCTCTC | SEQ ID NO: 243 | 24 | 13 |
| p13M-8 | Mini D13S1304-R | | ACATTCTAGTGCTACAGGGTATTC | SEQ ID NO: 244 | 24 | 13 |
| p13M-9 | Mini D13S289-F | * | GTCCCACTATCTCAATAATCTGATG | SEQ ID NO: 245 | 25 | 13 |
| p13M-10 | Mini D13S289-R | | ACTGGTCACCTTCATCACCA | SEQ ID NO: 246 | 20 | 13 |
| p13M-11 | Mini D13S171-F | * | GGAGAAAGGGGAGGTGTAGA | SEQ ID NO: 247 | 20 | 13 |
| p13M-12 | Mini D13S171-R | | CCATCCTCCTCCCTTCTTTTT | SEQ ID NO: 248 | 21 | 13 |
| p13M-13 | Mini D13S219-F | * | TTGCCATGTCAATTGCTACA | SEQ ID NO: 249 | 20 | 13 |
| p13M-14 | Mini D13S219-R | | TGTTTCTTGACTTAACATTTTCTTCT | SEQ ID NO: 250 | 26 | 13 |
| p13M-15 | Mini D13S218-F | * | GATTTGAAAATGAGCAGTCCA | SEQ ID NO: 251 | 21 | 13 |
| p13M-16 | Mini D13S218-R | | GCATGTTTCAGGTCTTTTATTGC | SEQ ID NO: 252 | 23 | 13 |
| p13M-17 | Mini D13S263-F | * | GCCTGTTAGTTTTTATTGTTATCTTAG | SEQ ID NO: 253 | 27 | 13 |
| p13M-18 | Mini D13S263-R | | TTTTTATCAGAAGCATGAAAACAG | SEQ ID NO: 254 | 24 | 13 |
| p13M-19 | Mini D13S153-F | * | CTGTTTCTCCTCCCTGCAAC | SEQ ID NO: 255 | 20 | 13 |
| p13M-20 | Mini D13S153-R | | GGAGCGTATCTGTGCGTGTA | SEQ ID NO: 256 | 20 | 13 |
| p13M-21 | Mini D13S1320-F | * | CACTTCTAGGTTTTTACCCAAGTGA | SEQ ID NO: 257 | 25 | 13 |
| p13M-22 | Mini D13S1320-R | | TGAAGTAACTCTGAACACTCAATACTT | SEQ ID NO: 258 | 27 | 13 |
| p13M-23 | Mini D13S1296-F | * | GTTTAACCAGGAGCCCTTCC | SEQ ID NO: 259 | 20 | 13 |
| p13M-24 | Mini D13S1296-R | | GAGCAACTACCTACTATGGTTCCTT | SEQ ID NO: 260 | 25 | 13 |
| p13M-25 | Mini D13S156-F | * | ACTCCAGCCTGGGCGATAG | SEQ ID NO: 261 | 19 | 13 |
| p13M-26 | Mini D13S156-R | | CTTGGATTTATGTATCTCTCCTAGAGT | SEQ ID NO: 262 | 27 | 13 |
| p13M-27 | Mini D13S1306-F | * | GCTGTTCTTCTAAGTGCCACA | SEQ ID NO: 263 | 21 | 13 |
| p13M-28 | Mini D13S1306-R | | GGGGTTGTTGCGAAGATTAG | SEQ ID NO: 264 | 20 | 13 |
| p13M-29 | Mini D13S170-F | * | TGGAGATAAACACATAGGCACA | SEQ ID NO: 265 | 22 | 13 |
| p13M-30 | Mini D13S170-R | | TAAGGCAGGAGTCATGTCCA | SEQ ID NO: 266 | 20 | 13 |
| p13M-31 | Mini D13S265-F | * | GCCAATTACATTGCATATTGCAT | SEQ ID NO: 267 | 23 | 13 |
| p13M-32 | Mini D13S265-R | | CAACAAAGCAATAAAGAGTTTTGC | SEQ ID NO: 268 | 24 | 13 |
| p13M-33 | Mini D13S1241-F | * | ATGGAGTGCCACTGGAAGAA | SEQ ID NO: 269 | 20 | 13 |
| p13M-34 | Mini D13S1241-R | | CCAGTTGAGTTTGGACCTCAG | SEQ ID NO: 270 | 21 | 13 |
| p13M-35 | Mini D13S159-F | * | GGCCAAAATTAGCGTGACA | SEQ ID NO: 271 | 19 | 13 |
| p13M-36 | Mini D13S159-R | | CAACTCCAGGCCAAATCATC | SEQ ID NO: 272 | 20 | 13 |
| p13M-37 | Mini D13S158-F | * | CGGAGTGAAAGAAGATTGATTT | SEQ ID NO: 273 | 22 | 13 |
| p13M-38 | Mini D13S158-R | | TTGACAATTTAGCAGCATGTATTT | SEQ ID NO: 274 | 24 | 13 |
| p13M-39 | Mini D13S173-F | * | AGCCTCATGCCTGGGGATA | SEQ ID NO: 275 | 19 | 13 |
| p13M-40 | Mini D13S173-R | | ATTTTCTTCATTTGGTGTTATTTTGG | SEQ ID NO: 276 | 26 | 13 |
| p13M-41 | Mini D13S1265-F | * | CTTTTCAGATTAAATGAGACAATATG | SEQ ID NO: 277 | 26 | 13 |
| p13M-42 | Mini D13S1265-R | | TGCTAATGTGTGATTATATGTACGC | SEQ ID NO: 278 | 25 | 13 |

Note:
* = 6-FAM, VIC, NED, JOE, ROX, PET, TAMRA or 5-FAM

TABLE 4

Mini-STR Primers for Chromosome 18

| | | | | | | |
|---|---|---|---|---|---|---|
| Number | Name/Loci | 5′ Label | Sequence | Seq. ID No. | Length | Chromosome |

| | | | | | | |
|---|---|---|---|---|---|---|
| p18M-1 | Mini D18S51-F | * | TGAGTGACAAATTGAGACCTT | SEQ ID NO: 279 | 21 | 18 |
| p18M-2 | Mini D18S51-R | | GTCTTACAATAACAGTTGCTACTATT | SEQ ID NO: 280 | 26 | 18 |
| p18M-3 | Mini D18S59-F | * | AACAGGGGCACAAGACAGAT | SEQ ID NO: 281 | 20 | 18 |
| p18M-4 | Mini D18S59-R | | CCCACTCCTGTGCACTCTCT | SEQ ID NO: 282 | 20 | 18 |
| p18M-5 | Mini D18S476-F | * | GTTGACAATAGCACACATACAGTCC | SEQ ID NO: 283 | 25 | 18 |
| p18M-6 | Mini D18S476-R | | ACCACCACCCACACCATC | SEQ ID NO: 284 | 18 | 18 |
| p18M-7 | Mini D18S63-F | * | TCTTCCATTCCCATCATTTCA | SEQ ID NO: 285 | 21 | 18 |
| p18M-8 | Mini D18S63-R | | TCTCCAGGAACATTGTTTTACTTT | SEQ ID NO: 286 | 24 | 18 |
| p18M-9 | Mini D18S1132-F | * | CAGCCTTTTCCAAATTTTACATC | SEQ ID NO: 287 | 23 | 18 |
| p18M-10 | Mini D18S1132-R | | TGCCCTACCAGACCAATTTT | SEQ ID NO: 288 | 20 | 18 |
| p18M-11 | Mini D18S452-F | * | TGGGGCATACATAGTGCAAA | SEQ ID NO: 289 | 20 | 18 |
| p18M-12 | Mini D18S452-R | | AAATAACCGCTGGCTCTGTG | SEQ ID NO: 290 | 20 | 18 |
| p18M-13 | Mini D18S464-F | * | GACTTTGTGCCATTTCTCCA | SEQ ID NO: 291 | 20 | 18 |
| p18M-14 | Mini D18S464-R | | AATCTTCAGGCTGCTTGCAC | SEQ ID NO: 292 | 20 | 18 |
| p18M-15 | Mini D18S1150-F | * | GGCACAGGAAACGTGAATTT | SEQ ID NO: 293 | 20 | 18 |
| p18M-16 | Mini D18S1150-R | | GCTGTTTTCTTCTGTTGTCTGG | SEQ ID NO: 294 | 22 | 18 |
| p18M-17 | Mini D18S53-F | * | TTTGGATGTCTTTCTTTCTTCTATCA | SEQ ID NO: 295 | 26 | 18 |
| p18M-18 | Mini D18S53-R | | CAGTGGAAACCAAACTACAACG | SEQ ID NO: 296 | 22 | 18 |
| p18M-19 | Mini D18S453-F | * | CAATAAAGACCTGACTTGGAAAAA | SEQ ID NO: 297 | 24 | 18 |
| p18M-20 | Mini D18S453-R | | CTCAACACAGCAACAAAAATAAA | SEQ ID NO: 298 | 25 | 18 |
| p18M-21 | Mini D18S478-F | * | AAGAGAAGAACATCACTAAGAACCA | SEQ ID NO: 299 | 25 | 18 |
| p18M-22 | Mini D18S478-R | | AACTCAGTGTTCCACAGTAACTC | SEQ ID NO: 300 | 24 | 18 |
| p18M-23 | Mini D18S56-F | * | CTGAAGGACCTGCCTGAGAT | SEQ ID NO: 301 | 20 | 18 |
| p18M-24 | Mini D18S56-R | | TACTTTTTATTGTTAGGGTGTGCTC | SEQ ID NO: 302 | 25 | 18 |
| p18M-25 | Mini D18S468-F | * | CCCCTGCATAAACTCACTCA | SEQ ID NO: 303 | 20 | 18 |
| p18M-26 | Mini D18S468-R | | TTCCAAAGGACATAATCCATATTT | SEQ ID NO: 304 | 24 | 18 |
| p18M-27 | Mini D18S450-F | * | GGACCTAGGTTCCAATTTCTCC | SEQ ID NO: 305 | 22 | 18 |
| p18M-28 | Mini D18S450-R | | TGTATGGTGCATGGAACCTGTG | SEQ ID NO: 306 | 21 | 18 |
| p18M-29 | Mini D18S474-F | * | CTGGCCTCCACCCACTAGAT | SEQ ID NO: 307 | 20 | 18 |
| p18M-30 | Mini D18S474-R | | CTTTCAATGTCAGAAGGCATTT | SEQ ID NO. 308 | 22 | 18 |
| p18M-31 | Mini D18S1127-F | * | ACCCTGGAGAGTGACTGCAT | SEQ ID NO: 309 | 20 | 18 |
| p18M-32 | Mini D18S1127-R | | CGCCTGTACTGCCTGAGTTT | SEQ ID NO: 310 | 20 | 18 |
| p18M-33 | Mini D18S1129-F | * | GGCTGCACAGGCATTC | SEQ ID NO: 311 | 16 | 18 |
| p18M-34 | Mini D18S1129-R | | GGGAATGCAGTGAAATGGAC | SEQ ID NO: 312 | 20 | 18 |
| p18M-35 | Mini D18S64-F | * | TTTTGCCACAAAAATTACCAA | SEQ ID NO: 313 | 21 | 18 |
| p18M-36 | Mini D18S64-R | | AAATCAGGAAATCGGCACTG | SEQ ID NO: 314 | 20 | 18 |
| p18M-37 | Mini D18S1147-F | * | TCAGCACAATGCTACTGGGTA | SEQ ID NO: 315 | 21 | 18 |
| p18M-38 | Mini D18S1147-R | | GACTGGGAACATGGCTCTTC | SEQ ID NO: 316 | 20 | 18 |
| p18M-39 | Mini D18S68-F | * | TGTGGAAAGTTGTAGATAGGATGAA | SEQ ID NO: 317 | 25 | 18 |
| p18M-40 | Mini D18S68-R | | TGAGGATCACACTTTGAGTAGTAAGTC | SEQ ID NO: 318 | 27 | 18 |
| p18M-41 | Mini D18S61-F | * | CCAAACTACATTCTTCTTCCTGA | SEQ ID NO: 319 | 23 | 18 |
| p18M-42 | Mini D18S61-R | | GAGGAATTTATGCTAAGATTTGAAGG | SEQ ID NO: 320 | 26 | 18 |
| p18M-43 | Mini D18S469-F | * | AACACGCTTGTCAAATGCTT | SEQ ID NO: 321 | 20 | 18 |
| p18M-44 | Mini D18S469-R | | TTAAGTTATTGTTGTTGTTTCTTGTGG | SEQ ID NO: 322 | 27 | 18 |
| p18M-45 | Mini D18S462-F | * | CAGAAGCAGATTTGAACATTGG | SEQ ID NO: 323 | 22 | 18 |
| p18M-46 | Mini D18S462-R | | GCTATAAACATTCACCGTTAGGG | SEQ ID NO: 324 | 23 | 18 |
| p18M-47 | Mini D18S70-F | * | GGCCTCTCTCCCAGAAAGAT | SEQ ID NO: 325 | 20 | 18 |
| p18M-48 | Mini D18S70-R | | TGTCAAGAAGTACCTACCATATTTTGA | SEQ ID NO: 326 | 27 | 18 |

Note:
* = 6-FAM, VIC, NED, JOE, ROX, PET, TAMRA or 5-FAM

TABLE 5

Mini-STR Primers for Chromosome X

| | | | | | | |
|---|---|---|---|---|---|---|
| Number | Name/Loci | 5′ Label | Sequence | Seq. ID No. | Length | Chromosome |

| | | | | | | |
|---|---|---|---|---|---|---|
| pXM-1 | Mini DXS1060-F | * | CTCCCTCTTAATGTTGCCTGT | SEQ ID NO: 327 | 21 | X |
| pXM-2 | Mini DXS1060-R | | TGAGAGTCTTTGGTGGGAGA | SEQ ID NO: 328 | 20 | X |
| pXM-3 | Mini DXS1223-F | * | TGCTTTTGGTGTCTTCAATCTG | SEQ ID NO: 329 | 22 | X |
| pXM-4 | Mini DXS1223-R | | TGGTCATGTAACAGTGCTTGG | SEQ ID NO: 330 | 21 | X |
| pXM-5 | Mini DXS8051-F | * | TGACATTTAATCAACCAAGAAAT | SEQ ID NO: 331 | 23 | X |
| pXM-6 | Mini DXS8051-R | | TTTTTGAACTAAGAACCTGGAG | SEQ ID NO: 332 | 22 | X |
| pXM-7 | Mini DXS7108-F | * | TTGTTAGTGTTTGCAAAGTGATGA | SEQ ID NO: 333 | 24 | X |
| pXM-8 | Mini DXS7108-R | | GGATTTATAGATATGGGAGGGTTC | SEQ ID NO: 334 | 24 | X |
| pXM-9 | Mini DXS1224-F | * | CCCTTGATGTAGGCACAGG | SEQ ID NO: 335 | 19 | X |
| pXM-10 | Mini DXS1224-R | | CGTGGGGGAGTAGTAGTGGT | SEQ ID NO: 336 | 20 | X |
| pXM-11 | Mini DXS8019-F | * | CTTCTTGCATTCCCCATGC | SEQ ID NO: 337 | 19 | X |
| pXM-12 | Mini DXS8019-R | | TTTCCTCACAGCAAAAGAGG | SEQ ID NO: 338 | 20 | X |
| pXM-13 | Mini DXS7593-F | * | CCTGGGCAACAAGAGTGAA | SEQ ID NO: 339 | 19 | X |
| pXM-14 | Mini DXS7593-R | | GGAAAGAGAGTTATATTTAAGAGCAGA | SEQ ID NO: 340 | 27 | X |
| pXM-15 | Mini DXS1226-F | * | CCCATCTGTCCTCCTGGATA | SEQ ID NO: 341 | 20 | X |
| pXM-16 | Mini DXS1226-R | | GGTCCCCTATTTGTCTTGTCC | SEQ ID NO: 342 | 21 | X |
| pXM-17 | Mini DXS1061-F | * | TCCCTTTCTCTCTCTCTCTCTCTC | SEQ ID NO: 343 | 24 | X |
| pXM-18 | Mini DXS1061-R | | TGATGTGTTATGAATTGGCAAAA | SEQ ID NO: 344 | 23 | X |
| pXM-19 | Mini DXS1214-F | * | GGTTGGAATGACTGAAGGCTTA | SEQ ID NO: 345 | 22 | X |
| pXM-20 | Mini DXS1214-R | | AAGATAGCAGGCAACAATAAGAT | SEQ ID NO: 346 | 23 | X |
| pXM-21 | Mini DXS1068-F | * | GGTTCTAGGGACACTCCCTTC | SEQ ID NO: 347 | 21 | X |
| pXM-22 | Mini DXS1068-R | | AGACCATGGCCTGCTTTTA | SEQ ID NO: 348 | 19 | X |
| pXM-23 | Mini DXS8015-F | * | GCCTTACACACAAGCACACC | SEQ ID NO: 349 | 20 | X |
| pXM-24 | Mini DXS8015-R | | GCACCAATATCAAAGCAGCA | SEQ ID NO: 350 | 20 | X |
| pXM-25 | Mini DXS993-F | * | ACCACTCAGCCAGTTTGCTT | SEQ ID NO: 351 | 20 | X |
| pXM-26 | Mini DXS993-R | | GAACTGGCCTTGCCTTCAC | SEQ ID NO: 352 | 19 | X |
| pXM-27 | Mini DXS8080-F | * | GGGCAACAAGAGCAAAACTC | SEQ ID NO: 353 | 20 | X |
| pXM-28 | Mini DXS8080-R | | CCCTGTTGGTAAATCCTTGG | SEQ ID NO: 354 | 20 | X |
| pXM-29 | Mini DXS8083-F | * | CAAGGAACTCAAACAACAGTTTACA | SEQ ID NO: 355 | 25 | X |
| pXM-30 | Mini DXS8083-R | | TCTTTGCCCACTTTTTAATGG | SEQ ID NO: 356 | 21 | X |
| pXM-31 | Mini DXS991-F | * | GGTTCTCCAGAGGGACAGAA | SEQ ID NO: 357 | 20 | X |
| pXM-32 | Mini DXS991-R | | TCTCCCTGATAAACTCCTTTTCAT | SEQ ID NO: 358 | 24 | X |
| pXM-33 | Mini DXS1216-F | * | TCTCTTTCAGTGACCCCTCCT | SEQ ID NO: 359 | 21 | X |
| pXM-34 | Mini DXS1216-R | | GGGGAAAGAGAGAGAGAGGAA | SEQ ID NO: 360 | 21 | X |
| pXM-35 | Mini DXS986-F | * | CCACAAGCAGATAAAGAAAATGTG | SEQ ID NO: 361 | 24 | X |
| pXM-36 | Mini DXS986-R | | TCATTTTTATGGCCATGGTATGT | SEQ ID NO: 362 | 23 | X |
| pXM-37 | Mini DXS1196-F | * | TATTTCCCCCAGCACCCTTT | SEQ ID NO: 363 | 20 | X |
| pXM-38 | Mini DXS1196-R | | TTTCAGTAAAATCATACACCTTTAACA | SEQ ID NO: 364 | 27 | X |
| pXM-39 | Mini DXS1217-F | * | ATCTTTGGAGGGGAAGGAGT | SEQ ID NO: 365 | 20 | X |
| pXM-40 | Mini DXS1217-R | | GAAGTATCGTATCTGAATCCCGTA | SEQ ID NO: 366 | 24 | X |
| pXM-41 | Mini DXS8020-F | * | TTCAAAGAGCCCTCTGCTGT | SEQ ID NO: 367 | 20 | X |
| pXM-42 | Mini DXS8020-R | | ACAATTCTGTATAGACTTTGTGTGT | SEQ ID NO: 368 | 25 | X |
| pXM-43 | Mini DXS1106-F | * | TGAGAACTCCCTAAACAAAATGT | SEQ ID NO: 369 | 23 | X |
| pXM-44 | Mini DXS1106-R | | TTCCTTGAATGTAAGGATTAGGG | SEQ ID NO: 370 | 23 | X |
| pXM-45 | Mini DXS1059-F | * | TTTGCCTACCACGGTTGTCT | SEQ ID NO: 371 | 20 | X |
| pXM-46 | Mini DXS1059-R | | ACCCGTCGTGGTTGTGAT | SEQ ID NO: 372 | 18 | X |
| pXM-47 | Mini DXS8088-F | * | TCCTGTTTTCCAGTACCAGAAGT | SEQ ID NO: 373 | 23 | X |
| pXM-48 | Mini DXS8088-R | | GAGTCTATTAGGAGCACAAAAAGG | SEQ ID NO: 374 | 24 | X |
| pXM-49 | Mini DXS8055-F | * | TTGACTAGAAATGCTCCCTCAA | SEQ ID NO: 375 | 22 | X |
| pXM-50 | Mini DXS8055-R | | CAGGTTTCTGTGTGGACATTG | SEQ ID NO: 376 | 21 | X |
| pXM-51 | Mini DXS8064-F | * | ACTCCAGCCTGAGCAACAG | SEQ ID NO: 377 | 19 | X |
| pXM-52 | Mini DXS8064-R | | CATTGCTCCCCCAACAACT | SEQ ID NO: 378 | 19 | X |
| pXM-53 | Mini DXS8067-F | * | GAGGGCAACAGAGTGGAGAC | SEQ ID NO: 379 | 20 | X |
| pXM-54 | Mini DXS8067-R | | TGATTTTGTACACATTTATGGGGTAT | SEQ ID NO: 380 | 26 | X |
| pXM-55 | Mini DXS1001-F | * | CCTTCACATGTATCCCCAAA | SEQ ID NO: 381 | 20 | X |
| pXM-56 | Mini DXS1001-R | | TGAATGGATAAAGAAAATGTGGT | SEQ ID NO: 382 | 23 | X |
| pXM-57 | Mini DXS8009-F | * | AAACTGTGGAAATTGCTTCCAT | SEQ ID NO: 383 | 22 | X |
| pXM-58 | Mini DXS8009-R | | TCAACAAATTCCAGGTTATGTCA | SEQ ID NO: 384 | 23 | X |
| pXM-59 | Mini DXS1047-F | * | TTTTAAAAACTTCTACAATGAGCA | SEQ ID NO: 385 | 24 | X |
| pXM-60 | Mini DXS1047-R | | CCTAGGTAACATAGTGAGACCTTGTC | SEQ ID NO: 386 | 26 | X |
| pXM-61 | Mini DXS1062-F | * | ATGATGCCTGGCACACAGTA | SEQ ID NO: 387 | 20 | X |
| pXM-62 | Mini DXS1062-R | | AAGCACTTTGAATCATTTACGG | SEQ ID NO: 388 | 22 | X |
| pXM-63 | Mini DXS984-F | * | ACCCCCACCTCCCTGAAATA | SEQ ID NO: 389 | 20 | X |
| pXM-64 | Mini DXS984-R | | TGCCCTACTCCATTCCACAC | SEQ ID NO: 390 | 20 | X |
| pXM-65 | Mini DXS1205-F | * | CCACTTGTCCTCTTGCTACACA | SEQ ID NO: 391 | 22 | X |
| pXM-66 | Mini DXS1205-R | | TGGCTTAGAGTACTTTTTCACTGC | SEQ ID NO: 392 | 24 | X |
| pXM-67 | Mini DXS1227-F | * | TCCAAAATAACACTGAAACACG | SEQ ID NO: 393 | 22 | X |
| pXM-68 | Mini DXS1227-R | | AAGGGTTTACTCCCCCAAAA | SEQ ID NO: 394 | 20 | X |
| pXM-69 | Mini DXS8106-F | * | GGTATAAAACTGAACTCATCAGCA | SEQ ID NO: 395 | 24 | X |
| pXM-70 | Mini DXS8106-R | | AGCTGTAGAGTTGAGGAATGTTTTC | SEQ ID NO: 396 | 25 | X |
| pXM-71 | Mini DXS8043-F | * | AAACATTTGGTTAGGCTAATTTCTAT | SEQ ID NO: 397 | 26 | X |
| pXM-72 | Mini DXS8043-R | | AAACAAATGCGAATTTAAAAAGA | SEQ ID NO: 398 | 23 | X |
| pXM-73 | Mini DXS8045-F | * | GGAGATTTCTTCCTTGTTGCAC | SEQ ID NO: 399 | 22 | X |
| pXM-74 | Mini DXS8045-R | | GCTAGGCTGTGTGTGTCTGTG | SEQ ID NO: 400 | 21 | X |
| pXM-75 | Mini DXS998-F | * | AAAGGCAAAGAAAAACTGTTGC | SEQ ID NO: 401 | 22 | X |
| pXM-76 | Mini DXS998-R | | GATCATTCATATAACCTCAAAAGAACT | SEQ ID NO: 402 | 27 | X |
| pXM-77 | Mini DXS8069-F | * | GGCATCGTATTCATTGTTCCA | SEQ ID NO: 403 | 21 | X |
| pXM-78 | Mini DXS8069-R | | AGGTTCTTCCAAATTATTTTTGTG | SEQ ID NO: 404 | 24 | X |
| pXM-79 | Mini DXS1073-F | * | TGAAACACTGCTCCCCTTG | SEQ ID NO: 405 | 19 | X |
| pXM-80 | Mini DXS1073-R | | CCGAGTTATTACAAAGAAGCACA | SEQ ID NO: 406 | 23 | X |

Note:
* = 6-FAM, VIC, NED, JOE, ROX, PET, TAMRA or 5-FAM

Although the invention has been described with reference to the presently preferred embodiment, it should be understood that various modifications can be made without departing from the spirit of the invention. Accordingly, the invention is limited only by the following claims.

1. A method for conducting a genetic test of a fetus comprising
isolating a nucleic acid sample from a cervical mucus sample obtained from a female subject containing the fetus,
wherein the nucleic acid sample consisting essentially of polynucleotides in a size ranging from about 50 base pairs to about 300 base pairs and
wherein the result of a genetic test on the nucleic acid sample is indicative of a genetic composition of the fetus.

2. The method of claim 1, wherein the cervical mucus sample is obtained by transcervical swabs, endocervical lavage, cytobrush, aspiration, intrauterine lavage, or a combination thereof.

3. The method of claim 1, wherein the nucleic acid sample is isolated by size fractionation.

4. The method of claim 1, wherein the nucleic acid sample is a DNA sample or an RNA sample.

5. The method of claim 1, further comprising using the isolated nucleic acid sample to test for a genetic composition not uniquely associated with Y chromosome, wherein the genetic composition of the isolated nucleic acid sample is indicative of the genetic composition of the fetus.

6. The method of claim 5, wherein the genetic composition is selected from the group consisting of monosomy, partial monosomy, trisomy, partial trisomy, chromosomal translocation, chromosomal duplication, chromosomal deletion, and chromosomal inversion.

7. The method of claim 5, wherein the genetic composition is indicative of a disease condition selected from the group consisting of Down Syndrome, Edwards Syndrome, Patau Syndrome, Fragile X Syndrome, Turner Syndrome, Klinefelter's Syndrome, Triple X syndrome, XYY syndrome, Trisomy 8, Trisomy 16, Wolf-Hirschhorn Syndrome, and RhD Syndrome.

8. The method of claim 1, further comprising using the isolated nucleic acid sample as a template for a genetic test using an assay technology selected from the group consisting of PCR, real-time PCR, LCR, Q-B-replicase, SDA, RCA, TMA, LADA, MDA, and invader.

9. The method of claim 1, further comprising using the isolated nucleic acid sample to test the presence of an allele in the fetus, wherein the presence of the allele is based on the amplification of a nucleotide fragment using a pair of primers specific for the allele.

10. The method of claim 9, wherein the allele corresponds to a genetic condition selected from the group consisting of sickle-cell anemia, Phenylketonuria, Tay-Sachs disease, Cystic Fibrosis, beta-Thalassemia, Adrenal Hyperplasia, Fanconi Anemia, Spinal Muscularatrophy, Duchenne's Muscular Dystrophy, Huntington's Disease, Myotonic Dystrophy, Robertsonian translocation, Angelman syndrome, DiGeorge Syndrome, Tuberous Sclerosis, Ataxia Telangieltasia, and Prader-Willi syndrome.

11. The method of claim 1, further comprising using the isolated nucleic acid sample to determine the presence of a genetic marker in the fetus, wherein the determination is based on the amplification of a nucleotide fragment using a pair of primers specific for the genetic marker.

12. The method of claim 9, wherein the pair of primers is selected from the group consisting of primers of SEQ ID NOs: 1 and 2; SEQ ID NOs: 3 and 4; SEQ ID NOs: 5 and 6; SEQ ID NOs: 9 and 10; SEQ ID NOs: 11 and 12; and SEQ ID NOs: 13 and 14.

13. The method of claim 9, wherein the pair of primers is selected from the group consisting of primer sets listed in Tables 2, 3, 4 and 5.

14. The method of claim 11, wherein the pair of primers is selected from the group consisting of primers of SEQ ID NOs: 1 and 2; SEQ ID NOs: 3 and 4; SEQ ID NOs: 5 and 6; SEQ ID NOs: 9 and 10; SEQ ID NOs: 11 and 12; and SEQ ID NOs: 13 and 14.

15. The method of claim 11, wherein the pair of primers is selected from the group consisting of primer sets listed in Tables 2, 3, 4 and 5.

16. A genetic testing kit suitable for testing genetic composition of a fetus comprising a pair of primers suitable for amplifying a desired allele or genetic marker, wherein the amplified nucleotide fragment is less than about 200 base pairs and wherein the desired allele or genetic marker is not uniquely associated with Y chromosome.

17. The genetic testing kit of claim 16, wherein the pair of primers is selected from the group consisting of primers of SEQ ID NOs: 1 and 2; SEQ ID NOs: 3 and 4; SEQ ID NOs: 5 and 6; SEQ CD NOs: 9 and 10; SEQ ID NOs: 11 and 12; and SEQ ID NOs: 13 and 14.

18. The genetic testing kit of claim 16, wherein the pair of primers is selected from the group consisting of primer sets listed in Tables 2, 3, 4 and 5.

19. The genetic testing kit of claim 16, further comprising an instruction for using the pair of primers to test genetic composition of a fetus.

20. The genetic testing kit of claim 16, further comprising an instruction for using the pair of primers to test genetic composition of a fetus on an isolated DNA sample from a cervical mucus sample obtained from a female subject containing the fetus, wherein the DNA sample consisting essentially of polynucleotides in a size ranging from about 50 base pairs to about 200 base pairs.

21. A genetic testing kit suitable for testing genetic composition of a fetus comprising an isolated DNA sample from a cervical mucus sample obtained from a female subject containing the fetus, wherein the DNA sample consisting essentially of polynucleotides in a size ranging from about 50 base pairs to about 200 base pairs.

22. The genetic testing kit of claim 21, further comprising an instruction of using the DNA sample to test the genetic composition of the fetus.

23. The genetic testing kit of claim 21, further comprising an instruction of using the DNA sample to test the genetic composition of the fetus in combination with a pair of primers suitable for amplifying a desired allele or genetic marker, wherein the amplified nucleotide fragment is less than about 200 base pairs.

24. The genetic testing kit of claim 23, wherein the desired allele or genetic marker is not uniquely associated with Y chromosome.

25. An isolated DNA sample useful for genetic testing of a fetus obtained by isolating DNA fragments in a size ranging from about 50 base pairs to about 200 base pairs from a cervical mucus sample obtained from a female subject containing the fetus.

26. The isolated DNA sample of claim 25, wherein the sample is substantially free of non-nucleic acid components.

27. A method of isolating a fetal nucleic acid sample comprising:
isolating a nucleic acid sample consisting essentially of polynucleotides of about 50 base pairs to about 300 base pairs in length from a cervical mucus sample obtained from a female subject containing the fetus.
